(19) United States
(12) Patent Application Publication
(10) Pub. No.: US 2024/0010961 A1
(43) Pub. Date: Jan. 11, 2024

(54) MINI-ORGAN INSERT FOR IN VITRO CO-CULTURE STUDIES

(71) Applicant: The Regents of the University of California, Oakland, CA (US)

(72) Inventors: Alice SORAGNI, Los Angeles, CA (US); Peyton John TEBON, Los Angeles, CA (US)

(73) Assignee: The Regents of the University of California, Oakland, CA (US)

(21) Appl. No.: 18/037,653

(22) PCT Filed: Dec. 7, 2021
(86) PCT No.: PCT/US21/62256

§ 371 Date: May 18, 2023

Related U.S. Application Data
(60) Provisional application No. 63/122,275, filed on Dec. 7, 2020.

(51) Int. Cl. C12M 1/12; C12M 1/32; C12M 3/00

(52) U.S. Cl. CPC C12M 23/06; C12M 23/12; C12M 21/08

(57) ABSTRACT

In one embodiment, the present disclosure provides a tissue culture well insert for use with a multi-wells tissue culture plate. The cylindrical well insert is configured to be inserted into the tissue culture wells, wherein the well insert comprises a carrier or platform section on which liver cells or other cells are deposited, and the carrier or platform section is positioned in the tissue culture wells so as to be suspended off the bottom of the tissue culture wells. The well insert is useful for co-culturing multiple cell types to create in vitro biological systems. One specific application is high-throughput anti-tumor drug screening in which the liver cells or other cells carried by the carrier or platform section of the insert are co-cultured with tumor organoids deposited or bioprinted to the bottom of the tissue culture well.

### FIELD

The present disclosure relates in general to the field of in vitro biological systems and cancer therapeutics. In one embodiment, the present disclosure provides device and methods for biological investigations of cell co-culture systems and rapid high-throughput screening of cancer therapeutics.

### BACKGROUND

Several cancer therapies are delivered as prodrugs, requiring chemical modifications to become active anti-tumor compounds. These molecules are particularly difficult to study in vitro as many require metabolism by enzymes such as cytochromes P450 (CYPs) that are responsible for degrading the compounds for clearance. Several of these metabolites cannot be directly synthesized due to their instability and reactivity. Even those that can be produced are susceptible to deactivation by undesirable side reactions and may precipitate in solution. By developing ex vivo systems to metabolize therapeutics, one can circumvent the need to test liver-metabolized drugs in patient-derived xenograft (PDX) models. Although animal models provide accurate metabolomic profiles, they are also expensive, time-consuming, and limited by the tumorigenicity of the grafted cells, making them ill-suited for rapid screening.

Currently, there are limits in rapid identification of effective therapies to improve clinical outcomes for many cancer patients. For example, bone sarcoma is a rare family of over 40 cancer types that disproportionately effects younger patients. The majority of patients diagnosed are under the age of 45 with over one-quarter of cases impacting patients under 20. Although these cancers only represent 0.2% of the newly diagnosed cases in the United States each year (˜3,600 cases), they are expected to be responsible for over 2,000 patient deaths in 2021. Osteosarcoma (˜1000 cases/year), Ewing sarcoma (˜290 cases/year), chordoma (˜300 cases/year), and fibrosarcoma (˜150 cases/year) are four major types of bone sarcomas. The tumors are often identified by imaging and confirmed by diagnostic biopsy. At present, treatment plans are developed based on histology of the biopsy, which yields limited information on the molecular characteristics of the tumor.

The primary approach for treating, for example, bone sarcomas is surgical resection which, depending on tumor type, can be accompanied by neo-adjuvant and/or adjuvant chemotherapy. Due to a large number of clinically distinct tumor types and the relatively low number of patients with bone sarcomas overall, only a small set of frontline therapies have been extensively tested. The intrinsic difficulties in performing large clinical trials have left potentially useful drug candidates untested. Despite extensive efforts, overall five-year survival rates have not improved significantly over the past few decades and have plateaued around 60-80% for patients with localized tumors and 30% for patients with metastatic bone sarcomas.

Thus, there is a need to develop improved platforms and methods for high-throughput drug screening that enables functional screening of chemotherapeutics, biologic, and targeted agents that are for example metabolized and activated in the liver directly on patient-derived tumor specimens to increase the number of therapeutics that can be tested and expand the number of patients who can benefit from functional personalized medicine.

### SUMMARY

In one aspect, the present disclosure provides a device for use with a tissue culture plate comprising a plurality of tissue culture wells, the device comprising a cylindrical well insert configured to be inserted into the tissue culture wells, wherein the well insert comprises a rim section on one end of the insert and one or more carrier or platform sections on an opposite end of the insert, the rim and carrier(s) or platform(s) joined by one or more struts, wherein the carrier or platform section or sections are disposed around a perimeter of the opposite end, and the carrier or platform section or sections are positionable in the tissue culture wells so as to be suspended above the bottom of the tissue culture wells. In some embodiments, the carrier or platform sections are filled with cells (hereinafter, “carrier or platform cells”) such as liver or immune cells, and the carriers or platforms immersed in a well comprising medium and cells such as tumor cells present in suspension or printed within a hydrogel matrix on the bottom of the well (hereinafter “well cells”). In some embodiments, the effect of a tested agent on the well cells may be modulated by the carrier or platform. In some embodiments, the metabolic or other activity of the carrier or platform by modulating the effect of an agent on the well cells provides a better prediction of the activity of the agent in vivo where well cells and carrier or platform coexist (e.g., well cells from a tumor biopsy and carrier or platform from the liver).

In another embodiment, the present disclosure provides a system for use in screening candidate small molecules or biologics for activities against target cells such as tumor cells, the system comprising (i) a tissue culture plate comprising a plurality of tissue culture wells, and (ii) the device comprising the well insert as described herein. The device may be configured to fit into a 384-well, 96-well, 48-well, 24-well, 12-well or 6-well plate well, rest on the top rim, and suspend the carriers or platforms submerged within the culture medium. Cancer cells or normal cells may be in the tissue culture wells and liver-derived or other metabolically active cells in the device insert. Alternatively, cancer cells or normal cells may be in the device insert and liver-derived or other metabolically active cells in the tissue culture wells.

In another embodiment, the present disclosure provides a method for identifying therapeutic agents or combination thereof for treating a tumor in a patient, comprising the steps of obtaining a sample of tumor cells optionally in single cell suspension, or as aggregates or clusters from the tumor of the patient; dispensing geometrically-shaped organoid extrudates comprising the tumor cells into tissue culture wells or placing the tumor cells in medium in tissue culture wells; dispensing an active cell complex comprising active cells that can metabolize the therapeutic agent or combination thereof onto the carrier or platform section or sections of the device described herein; and inserting the well insert into the tissue culture wells, and culturing the tumor cells and the active cells in the tissue culture wells with a therapeutic agents or a combination thereof, wherein reduced growth or reduced mobility of the tumor cells in the presence of a therapeutic agent or combination thereof identifies the therapeutic agent or combination thereof for treating the tumor in the patient. In some embodiments, the presence of cells that can metabolize the therapeutic agent(s) provides an assay system that more closely mimics the disposition of the therapeutic agent in a patient, and in some embodiments provides an improved system for identifying potentially effective therapeutic agents.

In another embodiment, the present disclosure provides a method for treating a tumor in a patient, comprising the steps of obtaining a sample of tumor cells optionally in single cell suspension, or as aggregates or clusters from the tumor of the patient; dispensing shaped organoid extrudates comprising the tumor cells into tissue culture wells or tumor cells in medium in tissue culture wells; dispensing an active cell complex comprising active cells that can metabolize the therapeutic agent or combination thereof onto the carrier or platform vessel section or sections of the device described herein; inserting the well insert into the tissue culture wells, and culturing the tumor cells and the active cells in the tissue culture wells with the therapeutic agent or combination thereof, wherein reduced growth or reduced mobility of the tumor cells in the presence of the therapeutic agents or combination thereof identifies the therapeutic agents or combination thereof for treating the tumor in the patient; and treating the patient with the therapeutic agent or combination thereof. In some embodiments, the presence of cells that can metabolize the therapeutic agent(s) provides an assay system that more closely mimics the disposition of the therapeutic agent in a patient, and in some embodiments provides an improved system for identifying potentially effective patient treatments.

Thus, in one aspect, a device is provided for use with a tissue culture plate comprising a plurality of tissue culture wells, the device comprising a cylindrical well insert configured to be inserted into the tissue culture wells, wherein the well insert comprises a rim section on one end of the insert and one or more carrier or platform sections on an opposite end of the insert, wherein the carrier or platform section or sections is disposed around a perimeter of the opposite end, and the carrier or platform section or sections is positionable in the tissue culture wells so as to be suspended off the bottom of the tissue culture wells. In some embodiments, the tissue culture plate is a 384-well plate or a 96-well plate or a 48-well plate or a 24-well plate or a 12-well plate or an 8-well plate.

In some embodiments of the device, the distance between the rim section and the carrier or platform section is less than the depth of the tissue culture wells. In some embodiments, the carrier or platform section is suspended off the bottom of the tissue culture wells by about 1 mm. In some embodiments, the rim section has a diameter slightly larger than a diameter of the tissue culture wells.

In some embodiments of the device, the carrier or platform section is configured to accommodate a cell complex. In some embodiments, the cell complex is positioned not to occupy a central region of the tissue culture wells. In some embodiments, the carrier or platform section comprises one or more annular carriers or platforms. In some embodiments, the carrier or platform section comprises one annular carrier or platform. In some embodiments, the carrier or platform section comprises two concentric annular carriers or platforms. In some embodiments, the carrier or platform section comprises micropores and short vertical walls to keep the cell complex in place. In some embodiments, each of the micropores have a size of about 100 microns to about 1,000 microns.

In some embodiments, the cell complex comprises a hydrogel. In some embodiments, the cell complex comprises a basement membrane extract such as alginate, collagen, gelatin, Matrigel®, Cultrex® BME, CELLINK GelXA, CELLINK LAMININK 111, or any combination thereof.

In some embodiments, the well insert comprises a wall that comprises open lattice or does not comprise any open space.

In some embodiments, the carrier or platform section of the device disclosed herein comprises an active cell complex comprising cells that can metabolize one or more candidate therapeutic agents. In some embodiments, the active cell complex comprises liver cells. In some embodiments, the active cell complex comprises a hydrogel. In some embodiments, the active cell complex comprises basement membrane extract such as alginate, collagen, gelatin, Matrigel®, Cultrex® BME, CELLINK GelXA, CELLINK LAMININK 111, or any combination thereof.

In one aspect, a system is provided for use in screening candidate therapeutic agents for activities against tumor cells, the system comprising (i) a tissue culture plate comprising a plurality of tissue culture wells, and (ii) the device as herein before described. In some embodiments, the tissue culture plate is a 384-well plate or a 96-well plate or a 48-well plate or a 24-well plate or a 12-well plate or an 8-well plate. In some embodiments, the tissue culture wells comprises a tumor cell complex comprising tumor cells, the tumor cell complex is deposited as a shaped extrudate around a perimeter at a bottom of the tissue culture wells. In some embodiments, the tumor cells are sarcoma cells or carcinoma cells or normal tissue cells. In some embodiments, the tumor cell complex comprises a hydrogel. In some embodiments, the tumor cell complex comprises basement membrane extract such as alginate, collagen, gelatin, Matrigel®, Cultrex® BME, CELLINK GelXA, CELLINK LAMININK 111, or any combination thereof.

In some embodiments of the system herein, each of said devices comprises a tumor cell complex comprising tumor cells. In some embodiments, the tumor cells are sarcoma cells or carcinoma cells. In some embodiments, the tumor cell complex comprises a hydrogel. In some embodiments, the hydrogel is alginate, collagen, gelatin, or a basement membrane extract such as Matrigel®, Cultrex® BME, CELLINK GelXA, CELLINK LAMININK 111, or any combination thereof. In some embodiments, the tissue culture well comprises an active cell complex comprising cells that can metabolize one or more candidate therapeutic agents. In some embodiments, the active cell complex comprises liver cells. In some embodiments, the active cell complex comprises a hydrogel, such as alginate, collagen, gelatin, or a basement membrane extract such as Matrigel®, Cultrex® BME, CELLINK GelXA, CELLINK LAMININK 111, or any combination thereof.

In one aspect, a method is provided for identifying therapeutic agents or combination thereof for treating a tumor in a patient, comprising the steps of (i) obtaining a sample of tumor cells optionally in single cell suspension, or as aggregates or clusters from the tumor of the patient; (ii) dispensing shaped organoid extrudates comprising the tumor cells into tissue culture wells; (iii) dispensing an active cell complex comprising active cells that can metabolize the therapeutic agents or combination thereof onto the carrier or platform section of the device of claim 1; and (iv) inserting the device of claim 1 into the tissue culture wells, and culturing the tumor cells and the active cells in the tissue culture wells with the therapeutic agents or combination thereof, wherein reduced growth, reduced mobility, or reduced viability of the tumor cells in the presence of the therapeutic agents or combination thereof identifies the therapeutic agents or combination thereof for treating the tumor in the patient.

In one aspect, a method for treating a tumor in a patient, comprising the steps of (i) obtaining a sample of tumor cells optionally in single cell suspension, or as aggregates or clusters from the tumor of the patient; (ii) dispensing shaped organoid extrudates comprising the tumor cells into tissue culture wells; (iii)
dispensing an active cell complex comprising active cells that can metabolize the therapeutic agents or combination thereof onto the carrier or platform section of the device of claim 1; (iv) inserting the device of claim 1 into the tissue culture wells, and culturing the tumor cells and the active cells in the tissue culture wells with the therapeutic agents or combination thereof, wherein reduced growth, reduced mobility, or viability of the tumor cells in the presence of the therapeutic agents or combination thereof identifies the therapeutic agents or combination thereof for treating the tumor in the patient; and (v) treating the patient with the therapeutic agents or combination thereof.

In either of the foregoing aspects, in one embodiment, the dispensing of the shaped organoid extrudates or the active cell complex is independently performed by manual or automated bioprinting. In some embodiments, the tumor cells are sarcoma cells or carcinoma cells. In some embodiments, the shaped organoid extrudates comprise a hydrogel. In some embodiments, the shaped organoid extrudates comprise a basement membrane extract such as alginate, collagen, gelatin, Matrigel®, Cultrex® BME, CELLINK GelXA, CELLINK LAMININK 111, or any combination thereof. In some embodiments, the active cells comprise liver cells. In some embodiments, the active cell complex comprises a hydrogel. In some embodiments, the active cell complex comprises a basement membrane extract such as alginate, collagen, gelatin, Matrigel®, Cultrex® BME, CELLINK GelXA, CELLINK LAMININK 111, or any combination thereof.

In another aspect, a method is provided for identifying therapeutic agents or combination thereof for treating a tumor in a patient, comprising the steps of (i) obtaining a sample of tumor cells optionally in single cell suspension, or as aggregates or clusters from the tumor of the patient; (ii) providing the tumor cells into tissue culture wells; (iii) dispensing an active cell complex comprising active cells that can metabolize the therapeutic agents or combination thereof onto the carrier or platform section of the device of claim 1; (iv) introducing one or more therapeutic agents into the tissue culture wells, and culturing the tumor cells in the tissue culture wells with the therapeutic agents, wherein reduced growth, reduced mobility, or reduced viability of the tumor cells in the presence of the therapeutic agents identifies the therapeutic agents or combination thereof for treating the tumor in the patient.

In one aspect, a method for treating a tumor in a patient, comprising the steps of (i) obtaining a sample of tumor cells optionally in single cell suspension, or as aggregates or clusters from the tumor of the patient; (ii) providing the tumor cells into tissue culture wells; (iii) dispensing an active cell complex comprising active cells that can metabolize the therapeutic agents or combination thereof onto the carrier or platform section of the device of claim 1; (iv) introducing one or more therapeutic agents into the tissue culture wells, and culturing the tumor cells in the tissue culture wells with the therapeutic agents, wherein reduced growth, reduced mobility, or reduced viability of the tumor cells in the presence of the therapeutic agents identifies the therapeutic agents or combination thereof for treating the tumor in the patient; and (v) treating the patient with the therapeutic agents or combination thereof.

In either of the foregoing aspects, in one embodiment, the tumor cells are sarcoma cells or carcinoma cells. In some embodiments, the active cells comprise liver cells. In some embodiments, the active cell complex comprises a hydrogel. In some embodiments, the active cell complex comprises a basement membrane extract such as alginate, collagen, gelatin, Matrigel®, Cultrex® BME, CELLINK GelXA, CELLINK LAMININK 111, or any combination thereof.

In another aspect, a method is provided for identifying therapeutic agents or combination thereof for treating a tumor in a patient, comprising the steps of (i) obtaining a sample of tumor cells optionally in single cell suspension, or as aggregates or clusters from the tumor of the patient; (ii) dispensing said tumor cells onto the carrier or platform section of the device of claim 1; (iii) optionally dispensing an active cell complex comprising active cells that can metabolize said therapeutic agents or combination thereof into tissue culture wells; and (iv) inserting the device into said tissue culture wells, and culturing said tumor cells and optionally said active cells in said tissue culture wells with said therapeutic agents or combination thereof, wherein reduced growth, reduced mobility, altered biological properties or reduced viability of the tumor cells in the presence of said therapeutic agents or combination thereof identifies the therapeutic agents or combination thereof for treating the tumor in the patient.

In one aspect, a method is provided for treating a tumor in a patient, comprising the steps of (i) obtaining a sample of tumor cells optionally in single cell suspension, or as aggregates or clusters from the tumor of the patient; (ii) dispensing said tumor cells onto the carrier or platform section of the device of claim 1; (iii) optionally dispensing an active cell complex comprising active cells that can metabolize said therapeutic agents or combination thereof into tissue culture wells; (iv) inserting the device of claim 1 into said tissue culture wells, and culturing said tumor cells and optionally said active cells in said tissue culture wells with said therapeutic agents or combination thereof, wherein reduced growth, reduced mobility, altered biological properties, or viability of the tumor cells in the presence of said therapeutic agents or combination thereof identifies the therapeutic agents or combination thereof for treating the tumor in the patient; and (v) treating said patient with said therapeutic agents or combination thereof.

In the foregoing embodiments, the dispensing of the tumor cells or the active cell complex is independently performed by manual or automated bioprinting. In some embodiments, the tumor cells are sarcoma cells or carcinoma cells. In some embodiments, the tumor cells are provided in a hydrogel. In some embodiments, the hydrogel is alginate, collagen, gelatin, or a basement membrane extract such as Matrigel®, Cultrex® BME, CELLINK GelXA, CELLINK LAMININK 111, or any combination thereof. In some embodiments, the active cells comprise liver cells. In some embodiments, the active cell complex comprises a hydrogel. In some embodiments, the hydrogel is alginate, collagen, gelatin, or a basement membrane extract such as Matrigel®, Cultrex® BME, CELLINK GelXA, CELLINK LAMININK 111, or any combination thereof.

In one aspect, a system is provided for use in in vitro evaluation of a biological activities, the system comprising (i) a tissue culture plate comprising a plurality of tissue culture wells, and (ii) the device as described hereinabove.

These and other aspects will be appreciated from the ensuing descriptions of the figures and detailed description.

### BRIEF DESCRIPTION OF THE DRAWINGS

Some embodiments are herein described, by way of example only, with reference to the accompanying drawings. With specific reference now to the drawings in detail, it is stressed that the particulars shown are by way of example and for purposes of illustrative discussion of embodiments. In this regard, the description taken with the drawings makes apparent to those skilled in the art how embodiments may be practiced.

FIGS. 1A-1B present elevated perspective views of two embodiments of the mini organoid insert (MOI) device of the present disclosure. The depicted MOI devices comprise three vertical struts supporting one or more annular carriers or platforms. The MOI device may be configured to fit into a 384-well, 96-well, 24-well, 12-well or 6-well plate well, rest on the top rim, and suspend the carriers or platforms submerged within the culture medium. The embodied MOI devices, which may be 3D-printed, comprise one or more annular carriers or platforms at the bottom of the vertical struts, into such annular carriers or platforms “carrier or platform” (e.g., mammalian hepatocytes) may be seeded (in a hydrogel or other basement membrane extracts (BME) e.g., in alginate, collagen, gelatin, Matrigel® or other supports). The MOI device carrying the seeded “carrier or platform” is then inserted into a well-plate well, the base of the well already seeded with its own “well cells”, e.g., a 2D monolayer of cells, manually seeded 3D cell models or pre-printed shaped tumor organoid extrudate, and thus two cell lines may be conveniently co-cultured. Metabolic activity of mammalian liver cells often modulates the therapeutic activity of many pharmaceutical agents being tested on tumors, and thus co-culturing liver organoid carrier or platform together with tumor well cells simulate the bioactivity of the therapeutic agent in vivo. The MOI device may be suited to any organoid culture application. FIG. 1A presents an elevated perspective view of an MOI device having one basal annular carrier or platform on which a cell culture, such as a cell tissue culture, may be grown. The embodiment of FIG. 1A may be referred to as a “one annulus” embodiment. FIG. 1B presents an elevated perspective view of an MOI device having two concentric basal annular support carriers or platforms on which cell cultures, such as an organoid tissue cultures, may be grown. The two annuli of this embodiment may culture the same cell type or different cell types. The embodiment of FIG. 1B may be referred to as a “dual annulus” embodiment.

FIGS. 2A-2B present orthogonal overhead views of the one annulus embodiment, FIG. 2A, and dual annulus embodiment, FIG. 2B, of the MOI device of the present disclosure. Broken lines indicate transparency.

FIGS. 3A-3B present orthogonal side views of the one annulus embodiment, FIG. 3A, and dual annulus embodiment, FIG. 3B, of the MOI device of the present disclosure.

FIG. 4 presents a cross-sectional view of the one annulus embodiment of the present disclosure. Cross-hatching indicates sectioned solid surfaces. From the depicted perspective, one of the three vertical support structures is visible. The annular upper support is configured to fit over the opening of 384-well, 96-well, 24-well, 12-well or 6-well well plate openings. A cell culture such as an organoid, e.g., a mammalian liver organoid, may be cultured in the annulus-shaped void on the basal section of the MOI device. The cell culture may be suspended in this void space and held by adhesive and/or surface tension properties inherent to the support material containing cultured cells.

FIGS. 5A-5F depict various views of an embodiment of the MOI device of the present disclosure, the depicted embodiment comprising three vertical pillars, each pillar supporting a small fenestrated holding vessel. FIG. 5A depicts an elevated perspective view of the depicted embodiment of the MOI device. FIG. 5B depicts an alternate elevated perspective view of the depicted embodiment. FIG. 5C depicts a cross-sectional view of the depicted embodiment. FIG. 5D depicts an orthogonal side view of the depicted embodiment resting in a well of a well plate. FIG. 5E depicts an elevated perspective view, and zoomed inset view, of the depicted embodiment awaiting insertion into a well of a 96-well plate. FIG. 5F depicts a closeup elevated perspective view of the depicted embodiment awaiting insertion into a well of a 96-well plate.

FIGS. 6A-6B depict elevated perspective views of the one annulus and dual annulus embodiments resting in a well of a 96-well plate. FIG. 6A depicts the one annulus embodiment. FIG. 6B depicts both embodiments, each embodiment in its own well.

FIGS. 7A-7C depict side views of three embodiments of the MOI device resting in a well-plate well. FIG. 7A depicts a side view of either the one annulus embodiment or two annulus embodiment resting in a well-plate well. FIG. 7B depicts a side cross-sectional view of the one annulus embodiment. FIG. 7C depicts a side cross-sectional view of the two annulus embodiment.

FIG. 8 depicts a labeled side cross-sectional view of an embodiment of the MOI device of the present disclosure, the depicted embodiment comprising three vertical pillars, each pillar supporting a small fenestrated holding vessel.

FIGS. 9A-9C depict various views of an alternative fenestrated embodiment of the MOI device of the present disclosure, the depicted embodiment comprising three vertical pillars, each pillar supporting a small fenestrated holding vessel, the depicted embodiment having circular punch-hole fenestrations. FIG. 9A depicts an elevated perspective view. FIG. 9B depicts a side cross-sectional view of the embodiment resting in a well-plate well. FIG. 9C an elevated perspective view of the depicted embodiment awaiting insertion into a well of a 24-well plate.

FIG. 10A-10B depict various views of an embodiment of the MOI device. The depicted embodiment has six vertical struts holding a perforated ring carrier or platform base. Cultured cells, such as a mammalian liver organoids, may be dispersed in the perforated ring base. Culture medium may flow freely through the perforations dispersed around the basal surface of the ring base. FIG. 10A depicts an elevated perspective view of an embodiment of the MOI device. FIG. 10B depicts an elevated perspective view, and zoomed inset view, of the depicted embodiment awaiting insertion into a well of a 96-well plate.

FIGS. 11A-11B depict schematics of dual-cell cultures in a well-plate well. FIG. 11A depicts a dual culture wherein a Matrigel® ring (light gray) seeded with tumor cells (dark grey circles) is dispersed on the base of the well, and organoid cells, e.g., mammalian liver cells (black triangles) are suspended in culture medium. FIG. 11B depicts a dual culture employing one embodiment of the MOI device of the present disclosure, the MOI device inserted in a well-plate well, and with the basal portion of the MOI device submerged in culture medium. Matrigel® ring (light gray) seeded with tumor cells (dark grey circles) is dispersed on the base of the well. Organoid cells, e.g., mammalian liver cells (black triangles) are grown in the small fenestrated pockets located at the base of the MOI device. Culture medium is permitted to flow freely through the fenestrations. A metabolite, such as a prodrug, may be added to the culture medium, and the organoid may metabolize the metabolite, such that the effect of the metabolite on the tumor cells may be evaluated.

FIGS. 12A-12B depict control and experimental tumor cell viability data obtained using the MOI device of the present disclosure. Using the MOI device, tumor cells were co-cultured without, FIG. 12A, and with, FIG. 12B, liver organoid suspended on the MOI device. In the control, FIG. 12A, tumor cell viability was not significantly diminished by logarithmically increasing concentration of the chemotherapeutic drug cyclophosphamide (CP). In the presence of MOI-device-supported liver organoid, FIG. 12B, tumor cell viability decreased at higher CP concentrations.

FIG. 13A-C depicts elevated perspective views of multiple MOI devices connected to span multiple wells of a well plate. FIG. 13A depicts a linear 8-well embodiment. FIG. 13B depicts a linear 12-well embodiment. FIG. 13C depicts a two-dimensional 96-well array to be used with a 96-well plate.

### DETAILED DESCRIPTION

Provided herein is a device for use in in vitro biological systems for purposes including but not limited to cancer research, wherein any cell type(s) may be incorporated into in vitro biological culture systems. The device of the present disclosure enables the indirect co-culturing of cell populations or cell complexes in either two- or three-dimensional modalities without using artificially restrictive membranes or barriers. In some embodiments, the cell population or cell complex can comprise a 3D shape. In some embodiments the cell population or cell complex comprises essentially a 2D monolayer. In some embodiments the cell population or cell complex is a self-assembled spheroid/aggregate. The device disclosed herein is compatible with any cell-containing structure at the bottom of a well.

As will be described herein, the device disclosed herein provides one or more additional cell types that when co-cultured in the proximity of the cell population or cell complex, can provide further information on the culture system. In one embodiment, cells in the device are metabolically active, and may, in one embodiment, affect the activity of a compound being tested on the cell population or cell complex (e.g., liver cells that may metabolize and inactivate or activate a drug being screened for antitumor activity). In another embodiment, metabolically active cells in the cell population or cell complex may affect the activity of a test compound on cells present in the device. As will be described herein, any format in which at least one cell type or population is provided in, at or on the well bottom, and another at least one cell type or population is provided in the device carrier, platform or pocket, is embraced herein. In some embodiments, tumor cells are provided in the well and liver cells are provided in the device. In some embodiments, tumor cells are provided in the device and liver cells are provided in the well. In some embodiments the cells in the well are in culture medium, adherent to the well bottom, or both. In some embodiments the cells in the well are provided in a shaped organoid extrudate. In some embodiments different cell populations or cell complexes in the well are in an extrudate and free or adherent in culture.

In one embodiment, the present disclosure provides a well insert device 101 that is compatible with high-throughput drug screening methods using 384-well, 96-well, 48-well, 24-well, 12-well or 6-well culture plates. The mini-organ insert (MOI) well insert device comprises a carrier or platform or platforms 103 capable of holding a hydrogel or other matrix, in which cells 113, such as but not limited to liver cells or immune cells, can be placed and therein cultured. In one embodiment, the cells in the hydrogel metabolize compounds within the well 111, and in other embodiments, tumor or other cells are also present in the well, such as in shaped organoid extrudates 115 as described herein. In one embodiment, such tumor cells respond to the metabolized compounds and indicate whether the compound will, when administered to a patient who will similarly metabolize the compound, will be effective as a cancer chemotherapeutic agent.

The well insert device 101 is placed in a well 111 and hangs on the rim of the well to suspend the carrier or platform or platforms 103 of the device in the cell culture medium. The insert of the present disclosure may comprise micropores or perforations in the carrier or platform walls and/or bottom, and the carrier or platform or platforms 103 are dispersed around the perimeter of the device and thus proximal to the walls of the well 111, such that the center of the well 111 is unobstructed for changing medium, for example.

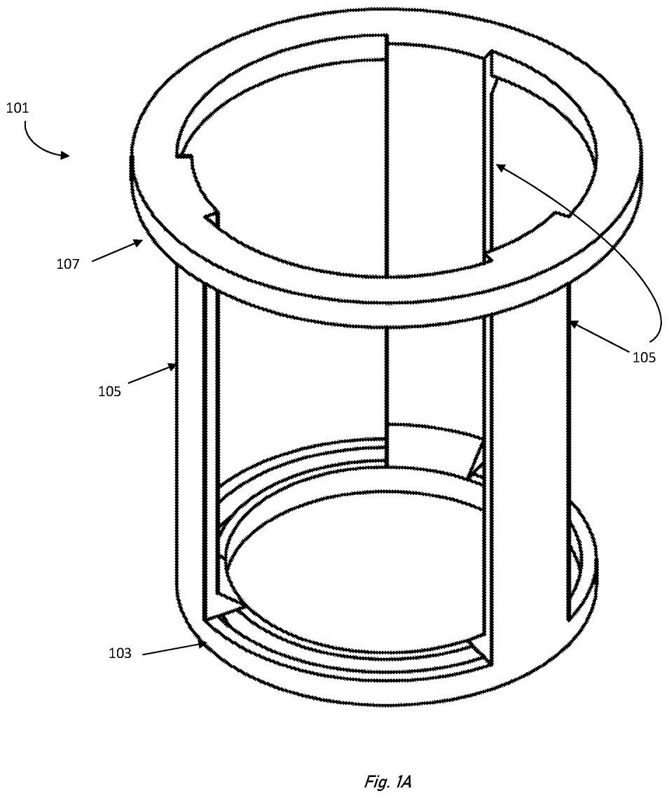

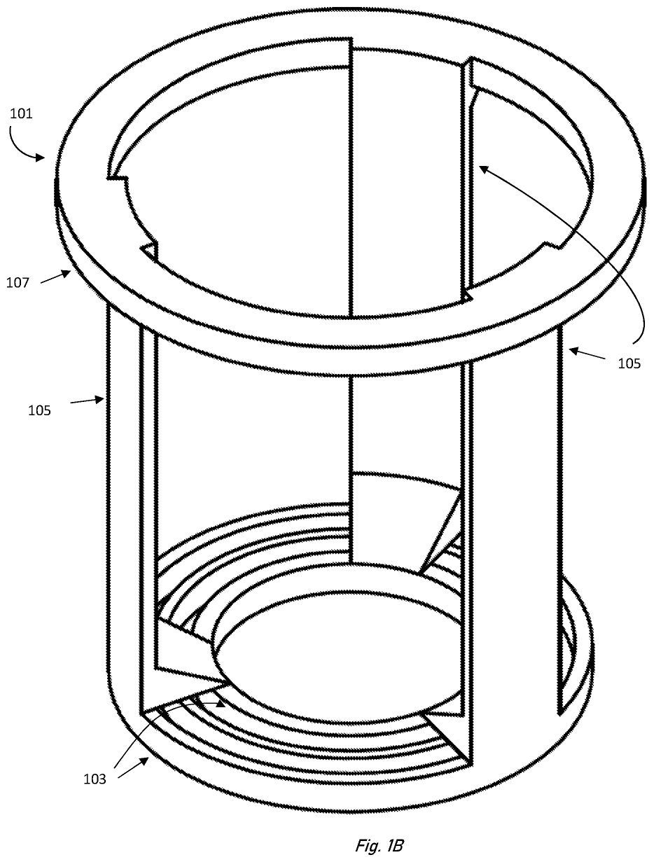

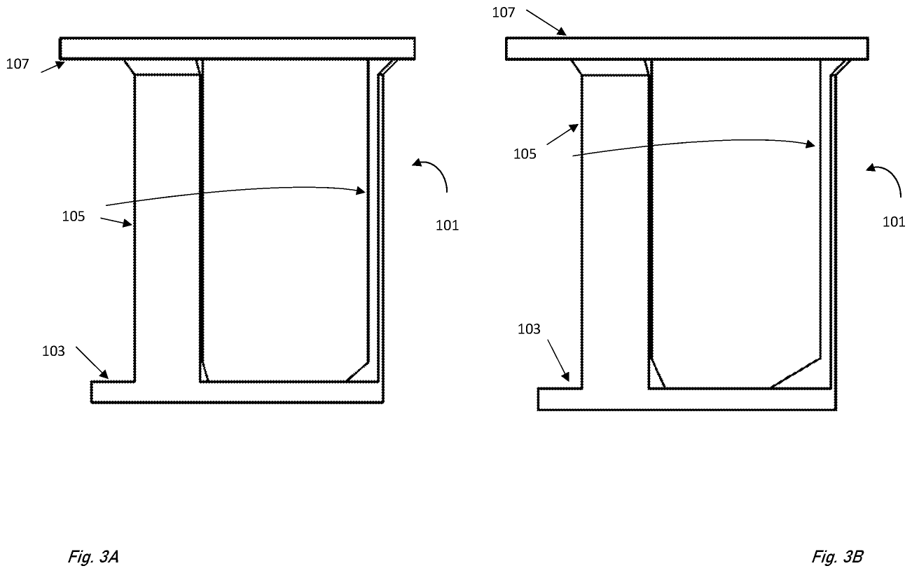

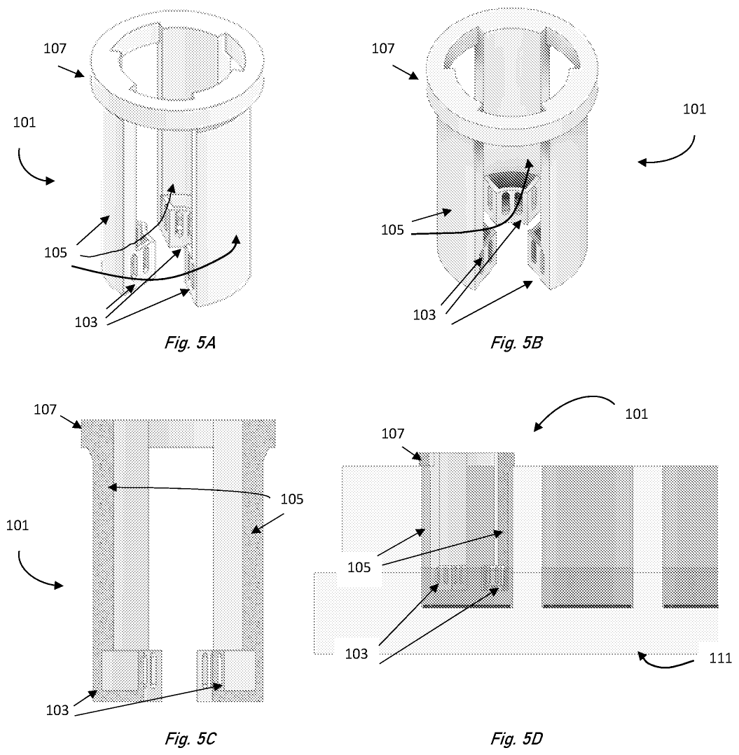

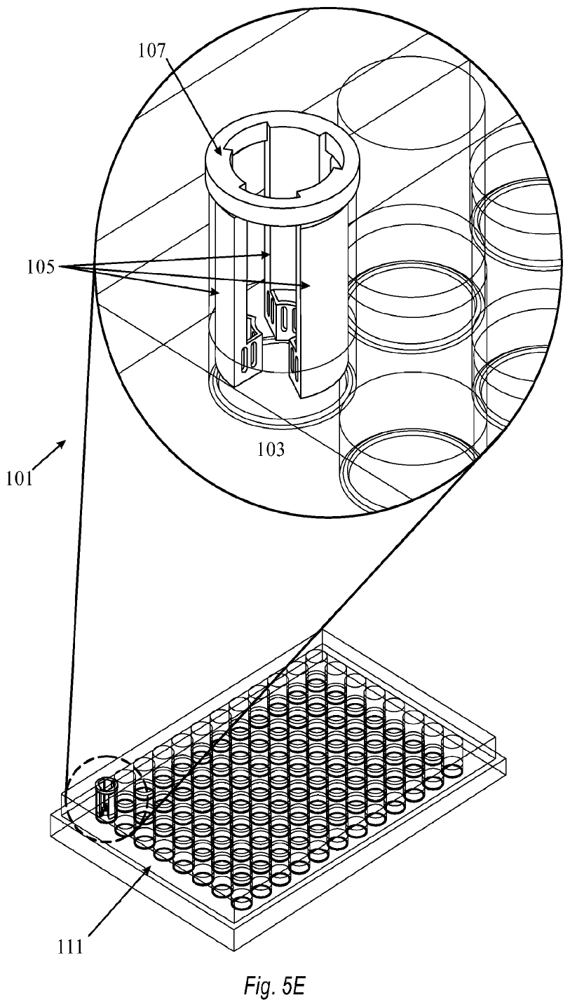

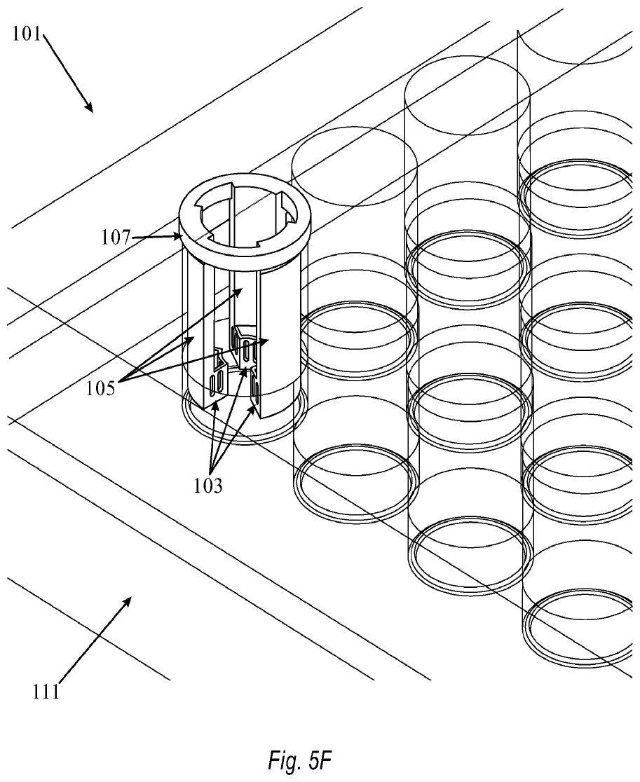

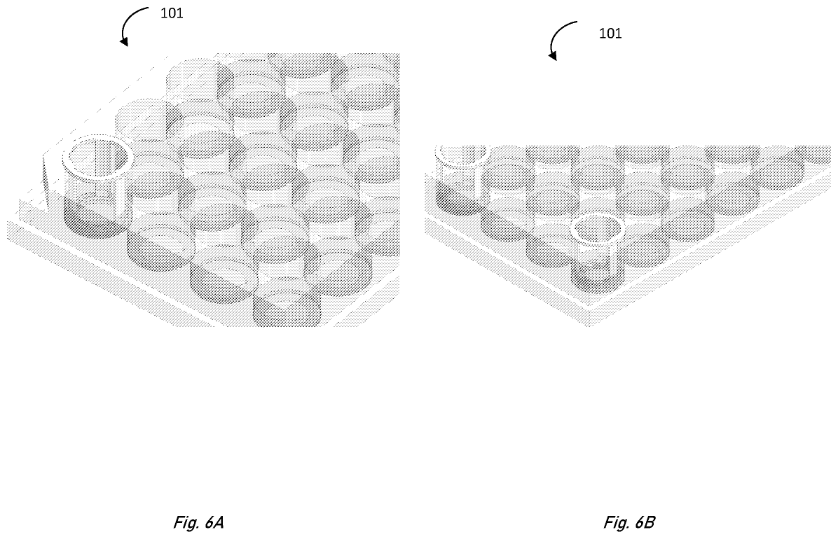

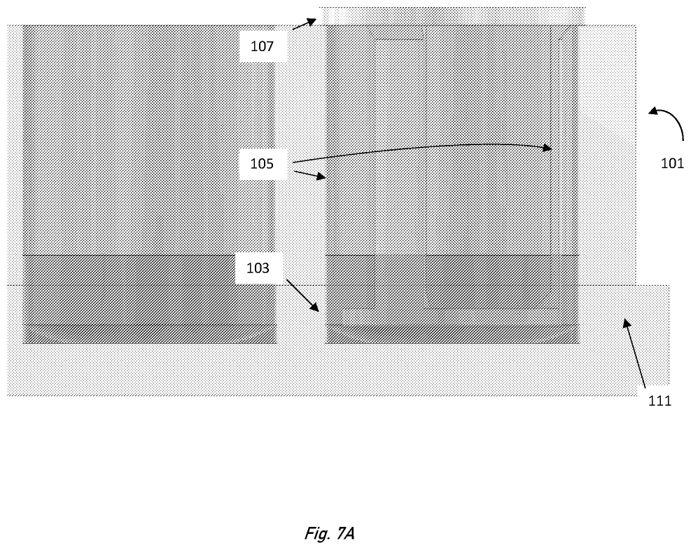

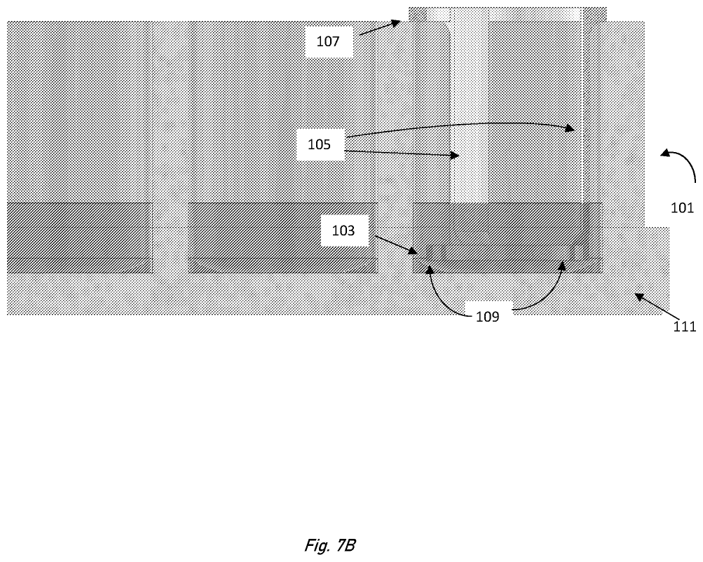

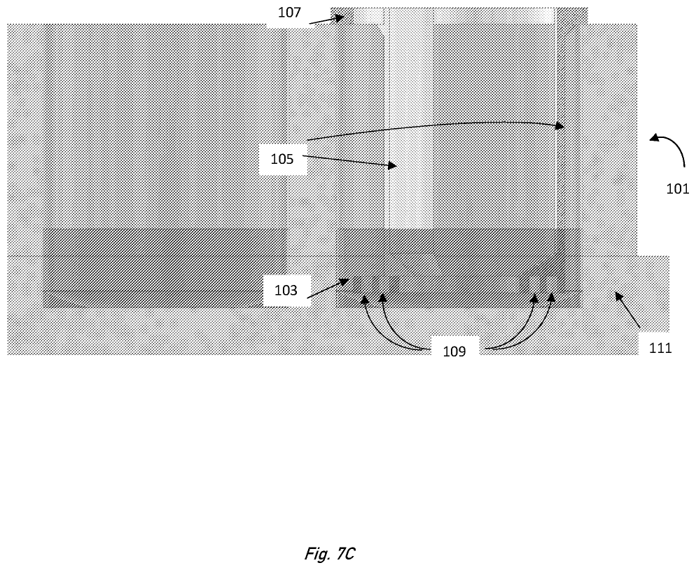

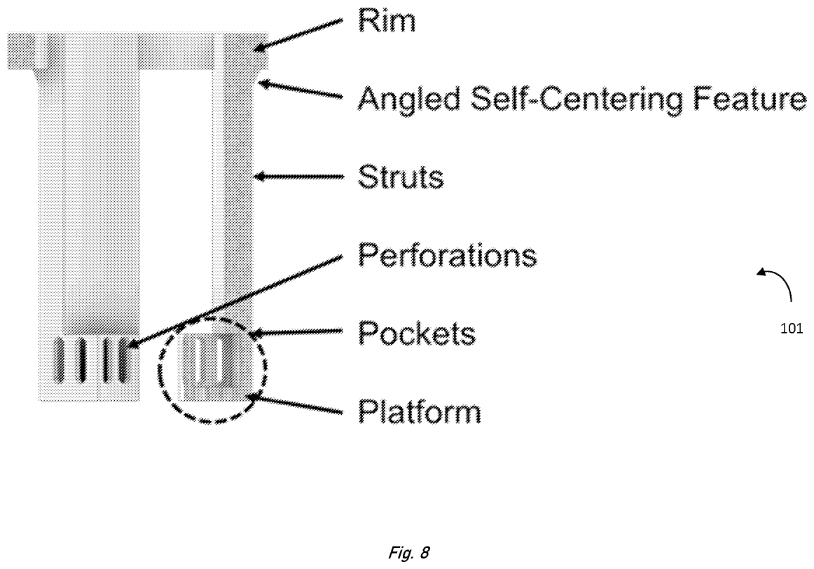

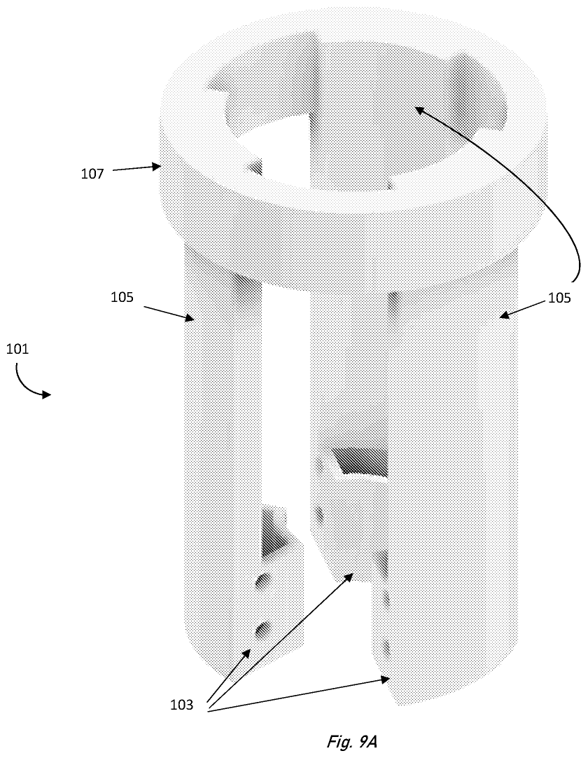

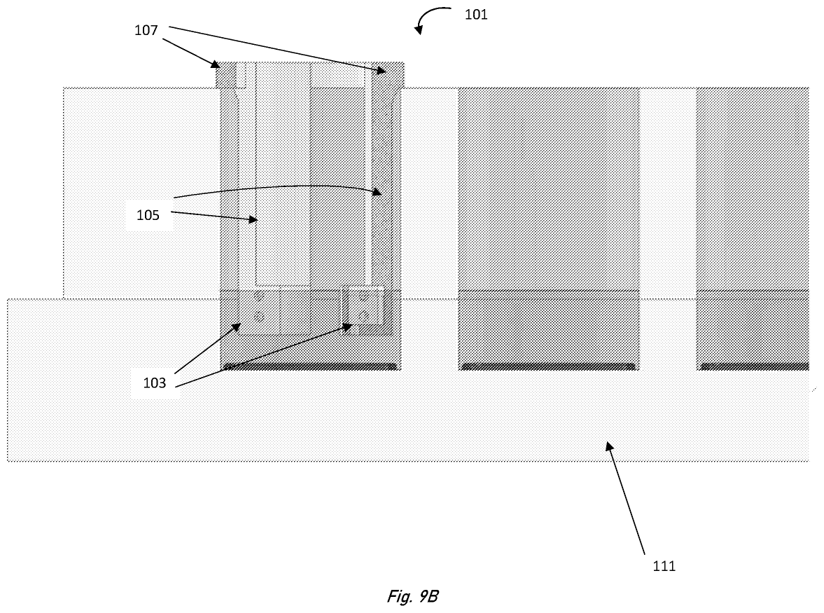

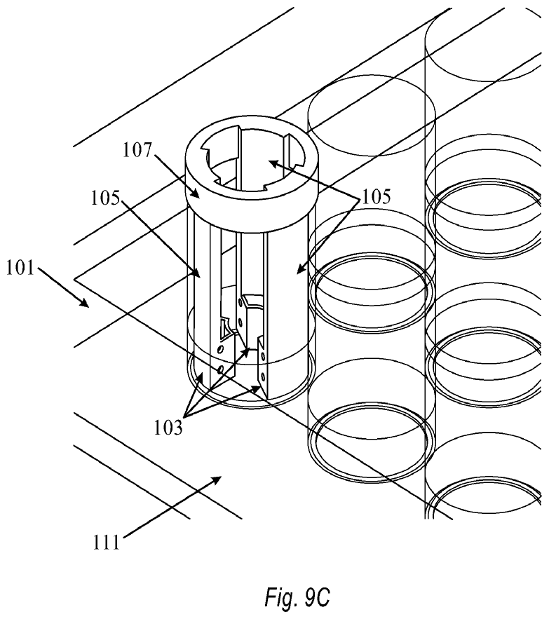

In one embodiment, the frame has a rim 107 with a diameter slightly larger than the diameter of the well 111 to prevent the insert from falling fully into the well. This rim supports the rest of the insert and ensures that the carrier or platform or platforms, the components supporting the hydrogel, is suspended off the bottom of the well. Non-limiting examples of inserts are shown in FIGS. 1A-1B, FIGS. 2A-2B, FIGS. 3A-3B, FIG. 4, FIGS. 5A-5F, FIG. 6A-6B, FIGS. 7A-7C, FIG. 8, FIGS. 9A-9C, and FIGS. 10A-10B.

The insert device 101 comprises the following components:
Carrier or platform. The carrier or platform 103 or carriers or platforms of the insert device comprise one or more small troughs or pockets 109 that are designed to hold a small volume of medium containing cells 113. The troughs may be perforated or fenestrated on the bottom and side walls such that exchange of agents and other components in the tissue culture medium may interact with the cells in the carrier or platform(s). In some designs of the insert, several carriers or platforms are provided, each suspended from the rim 107 from an individual strut 105. In some designs, a single, annular carrier or platform 103 is provided that is suspended from multiple struts 105. In some designs, multiple concentric annular carriers or platforms 103 are provided, the multiple concentric annular carriers or platforms suspended from multiple struts 105. The annular designs may have a bottom “floor” portion throughout the annulus, providing an enclosed annular trough 109. The floor portion may be perforated or fenestrated, permitting flow of culture medium. Alternatively, the annular carriers or platforms may be “open troughed”, having no floor portion. In such designs, organoids may be suspended in the annular carrier or platform open trough 109 by adhesion, surface tension, capillary action, or other natural forces between the material (e.g., tissue culture medium, BME, etc.) and the insert. The volume of the carrier or platform(s) may be adjusted to hold the desired volume of medium or hydrogel comprising cells. The carriers or platforms are positioned on the device to rest above the bottom of the well 111, so as not to interfere with any shaped organoid extrudate 115 or other solid or semisolid component 115 placed in the bottom of the well 111. Furthermore, the dimension of the carrier or platform that extends centrally is designed such that the core region of the well, i.e., the central region extending from the top to bottom, is not obstructed such that a needle or other sampling probe can readily be inserted into the well and not disturb the insert device or the cells on the carrier or platform(s). The term “carrier or platform” 103 may be used to refer to the carrier or platform 103 and pocket or trough 109 together. In one embodiment, the height of the pockets, carrier or platform may be between about 1 mm and 2 mm. In one embodiment the height of the pocket, carrier or platform is about 1 mm. In one embodiment the height of the pocket, carrier or platform is about 1.5 mm.
Struts. The struts 105 suspend the carrier or platform(s) from the rim 107. For a device with multiple individual carriers or platforms 103, each is suspended from the rim 107 with a strut 105. For a device with an annular carrier or platform, from one to multiple struts may be provided. The diameter of the disposition of the struts will be slightly smaller than the diameter of the well 111, such that when the insert is placed in a well, the struts are proximal to the wall of the well 111. For wells with a conical design or tapered walls, the struts may be designed to match the taper to permit facile insertion into the well 111. For insert devices used with wells with a small taper, the struts may be vertical.
Rim. The rim 107 of the device is slightly larger in diameter than the diameter of the well 111 to support the device on the top of the well and not allow it to fall fully into the well. The inner diameter of the rim provides a support for the struts that extend from the inner walls of the ring to the carrier or platform(s).

In one non-limiting example, for use in tissue culture plates with a well diameter of 6.86 mm at the top, 6.35 mm at the bottom, and a well depth of 1.5 cm, a rim has an outer diameter (OD) of 7.2 mm, the outer walls of the struts have an OD 6.3 mm. The top 0.5 mm of the struts have an angled feature to ensure self-centering. This region has a 6.3 mm diameter at the bottom and extends to an OD of 6.75 mm at the top. Generally, the manufacturing tolerances for both the plate and the inserts are high enough that the inserts rest against the walls of the wells near the bottom.

In one embodiment, the rim or portion of the struts adjacent to the rim are provided with angled features to ensure the insert is centered.

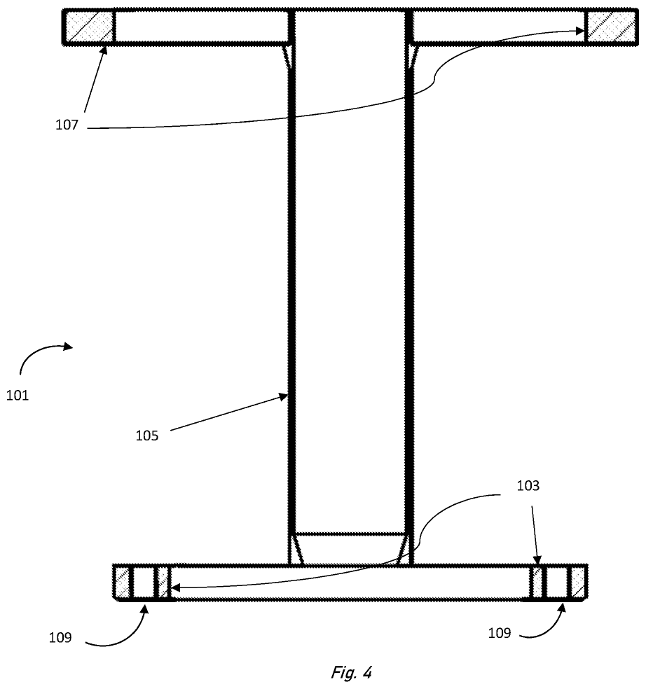

In some embodiments, the carrier or platform comprises multiple carriers or platforms each suspended from a strut from the rim. In some embodiments, the carrier or platform is an annulus- or ring-shaped section suspended using multiple struts. In any design of the carrier or platform, it comprises micropores (e.g. 100-1000 microns; also referred to herein as perforations or fenestrations) in the bottom surface of the carrier or platform and/or on its vertical walls (typically those walls in contact with the tissue culture medium, and not those walls at the periphery proximal to the well wall, but the disclosure is not so limiting) to allow for exchange of medium and the components therein between the tissue culture well and the cells disclosed upon the carrier or platform. In one embodiment, FIG. 4 depicts oblong perforations in the carrier or platform bottom and walls; the perforations in the bottom may be disposed concentrically a shown.

In one embodiment, the device, when used to provide liver cells in the well, directly immerses hydrogels containing hepatocytes into the culture medium so that multiple cell types share the same medium. This allows for better transfer of metabolized compounds within the medium without restricting diffusion through a membrane. Additionally, cells are kept away from the center of the well, making the device compatible with automated fluid handling protocols that use pipette tips for reaching the center of the wells. In one embodiment, the insert disclosed herein can be used in combination with high-throughput screening system disclosed herein to test compounds that are commonly used clinically, but difficult to study in vitro.

In one embodiment, the present disclosure provides a device for use with a tissue culture plate that contains a plurality of tissue culture wells, the device comprising a cylindrical well insert configured to be inserted into the tissue culture wells, wherein the well insert comprises a rim section on one end of the insert and a carrier or platform section on an opposite end of the insert, wherein the carrier or platform section is disposed around a perimeter of the opposite end, and the carrier or platform section is positionable in the tissue culture wells so as to be suspended off the bottom of the tissue culture wells.

In one embodiment, the tissue culture plate is a 384-well, 96-well plate, a 24-well plate, 12-well plate or a 6-well plate. In one embodiment, the distance between the rim section and the carrier or platform section of the well insert is less than the depth of the tissue culture wells. For example, the carrier or platform section is suspended off the bottom of the tissue culture wells by about 0.4 mm. In general, the rim section of the well insert has a diameter slightly larger than the diameter of the tissue culture wells. In one embodiment, sector-shaped or annular carrier or platform sections are configured to accommodate a cell complex (also referred to herein as a cell population). In one embodiment, the cell complex is positioned not to occupy a central region of the tissue culture wells. In another embodiment, the carrier or platform section comprises small pockets (e.g., having a size of about 100 microns to about 1,000 microns) and short vertical walls to keep the cell complex in place.

As noted herein, the device disclosed herein may comprise any type of cell useful for the various purposes herein. The device may comprise active cells that metabolize a drug being tested for activity against a tumor cells, the tumor cells provided in the wells (e.g., in culture or optionally as an organoid ring, etc.). The disclosure also embodies the reverse orientation thereof, where the active cells are provided in the well (e.g., in culture, or in a matrix, etc.), and the tumor cells are provided in the device. In another embodiment, the tumor cells are provided in the device, and a test compound or other conditions provided in the wells, without any cells provided in the wells, for example, screening test compounds or dilutions of a test compound against tumor cells or any other type of cell for in vitro screening purposes.

Thus, in one embodiment, the population of assay cells comprise two or more cell types, in separate locations or combined together. In one embodiment, the population of assay cells is provided in the tissue culture well. In one embodiment, the population of assay cells comprise two or more cell types, wherein at least one of the two or more cell types is provided in the well insert. In one further embodiment, a second cell type is provided in the well insert or in the tissue culture well. In one embodiment, at the at least two cell types are provided in the well insert. Thus, one or more assay cell types may be used as described in the disclosure herein, wherein each assay cell type may be combined with any other assay cell type, and each individual of mixed assay cell type provided in the well insert, in the tissue culture well, in both locations, and in some embodiments, one assay cell type may be provided in one carrier or platform of a well insert, and another assay cell type provided in the second carrier or platform of the same well insert. In some embodiments the same cell type may be provided in both carriers or platforms in the same well insert. In some embodiments, separate types of assay cells may be provided in each well insert carrier or platform and in the tissue culture well. Any combination or location or any one or more assay cell types is embraced herein.

In one embodiment, the cell complex deposited on the carrier or platform section comprises liver cells, or any other cells that can metabolize a therapeutic drug. In another embodiment, the cell complex deposited on the carrier or platform section comprises a hydrogel. For example, the cell complex comprises alginate, collagen, gelatin or a basement membrane extract (BME) sch as but not limited to Matrigel®, Cultrex® BME, CELLINK GelXA, CELLINK LAMININK 111, or any combination thereof.

In one embodiment, the cell-loaded hydrogel is deposited into the carrier or platform pockets using a pipette. In another embodiment, the cell-loaded hydrogel is deposited into the carrier or platform pockets using an automated fluid handling system or bioprinter. In another embodiment, the cell-loaded hydrogel is deposited into the carrier or platform pockets by submerging the pockets of the insert into a reservoir of liquid material. The submersion can occur once, or multiple times for durations from 0.5 to 10 seconds. Once the liquid hydrogel is loaded into the pockets, the material can be crosslinked with a thermal, photochemical, or chemical mechanism. In some embodiments, the loading of the pockets is performed at a temperature at which the hydrogel is liquid, e.g., at 2° C. When the loaded pockets are warmed to the incubation temperature of the plate, the hydrogel becomes a gel and traps the cells therein.

In one embodiment, the well insert comprises a wall that comprises open lattice. In another embodiment, the well insert comprises a wall that does not comprise any open space. As noted herein, the perforations or fenestrations in the carrier or platform bottom surface or walls may be of any shape and size to permit exchange of medium and components during operation, and to allow filling of the pocket with cell-loaded hydrogel without draining.

In another embodiment, the present disclosure provides a system for use in screening candidate therapeutic agents for activities against tumor cells, the system comprising (i) a tissue culture plate comprising a plurality of tissue culture wells, and (ii) the device comprising the well insert as described herein. In one embodiment, the tissue culture plate is a 384-well plate, 96-well plate, a 24-well plate, 12-well plate or a 6-well plate. In one embodiment, the tissue culture wells comprise a tumor cell complex comprising tumor cells, wherein the tumor cell complex is deposited as a ring around a perimeter at the bottom of the tissue culture wells. In other embodiments, the tumor cells are suspended in the tissue culture medium within the well. Any type of tumor or cancer can be deposited at the bottom of the tissue culture wells. Examples of tumor or cancer include, but are not limited to, carcinoma, sarcoma, lymphoma, leukemia, germ cell tumor, blastoma, chondrosarcoma, Ewing's sarcoma, malignant fibrous histiocytoma of bone, osteosarcoma, rhabdomyosarcoma, heart cancer, brain cancer, astrocytoma, glioma, medulloblastoma, neuroblastoma, breast cancer, medullary carcinoma, adrenocortical carcinoma, thyroid cancer, Merkel cell carcinoma, eye cancer, gastrointestinal cancer, colon cancer, gallbladder cancer, gastric (stomach) cancer, gastrointestinal carcinoid tumor, hepatocellular cancer, pancreatic cancer, rectal cancer, bladder cancer, cervical cancer, endometrial cancer, ovarian cancer, renal cell carcinoma, prostate cancer, testicular cancer, urethral cancer, uterine sarcoma, vaginal cancer, head cancer, neck cancer, nasopharyngeal carcinoma, hematopoietic cancer, Non-Hodgkin lymphoma, skin cancer, basal-cell carcinoma, melanoma, small cell lung cancer, non-small cell lung cancer, or any combination thereof.

In one embodiment, the tumor cell complex deposited at the bottom of the tissue culture wells comprises a hydrogel. For example, the tumor cell complex comprises alginate, collagen, gelatin or a basement membrane extract such as but not limited to Matrigel®, Cultrex® BME, CELLINK GelXA, CELLINK LAMININK 111, or any combination thereof. In one embodiment, the carrier or platform section of the well insert which is inserted into the tissue culture well comprises an active cell complex comprising cells that can metabolize one or more candidate therapeutic agents. For example, the active cell complex comprises liver cells. In another embodiment, the active cell complex comprises a hydrogel. For example, the active cell complex comprises Matrigel®, Cultrex® BME, CELLINK GelXA, CELLINK LAMININK 111, or any combination thereof.

In another embodiment, the present disclosure provides a method for identifying therapeutic agents or combination thereof for treating a tumor in a patient, comprising the steps of (i) obtaining a sample of tumor cells in single cell suspension from the tumor of the patient; (ii) dispensing shaped organoid extrudates comprising the tumor cells into tissue culture wells, or tumor cells within the medium in tissue culture wells; (iii) dispensing an active cell complex comprising active cells that can metabolize the therapeutic agents or combination thereof onto the carrier or platform section of the well insert disclosed herein; and (iv) inserting the well insert into the tissue culture wells, and culturing the tumor cells and the active cells in the tissue culture wells with the therapeutic agents or combination thereof, wherein reduced growth or reduced mobility of the tumor cells in the presence of the therapeutic agents or combination thereof identifies the therapeutic agents or combination thereof for treating the tumor in the patient. Examples of tumor or cancer have been described above. As noted herein, the presence of the active cells that metabolize a therapeutic agent mimics the disposition of the therapeutic agent in the patient wherein the active cells, such as liver cells or intestinal cells, may alter the therapeutic efficacy of a tested therapeutic agent. Use of the active cells, in the device as described herein or in any alternative forms, provides enhanced screening of potential therapeutically active compounds for a cancer patient. Such configuration of active cells together with target cells may also be used for other purposes wherein enhanced screening in vitro that utilizes multiple cell types that mimic in vivo conditions is desired.

In another embodiment, the present disclosure provides a method for treating a tumor in a patient, comprising the steps of (i) obtaining a sample of tumor cells optionally in single cell suspension, or as aggregates or clusters from the tumor of the patient; (ii) dispensing shaped organoid extrudates comprising the tumor cells into tissue culture wells or providing tumor cells suspended in medium; (iii) dispensing an active cell complex comprising active cells that can metabolize said therapeutic agents or combination thereof onto the carrier or platform section of the well insert disclosed herein; (iv) inserting the well insert into the tissue culture wells, and culturing the tumor cells and the active cells in the tissue culture wells with the therapeutic agents or combination thereof, wherein reduced growth or reduced mobility of the tumor cells in the presence of the therapeutic agents or combination thereof identifies the therapeutic agents or combination thereof for treating the tumor in the patient; and (v) treating the patient with the therapeutic agents or combination thereof. Examples of tumor or cancer have been described above. As noted herein, the presence of the active cells that metabolize a therapeutic agent mimics the disposition of the therapeutic agent in the patient wherein the active cells, such as liver cells, may alter the therapeutic efficacy of a tested therapeutic agent. Use of the active cells, in the device as described herein or in any alternative forms, provides enhanced identification of an effective cancer treatment regimen. Such configuration of active cells together with target cells may also be used for other purposes wherein enhanced identification of a therapeutic regimen is desired using in vitro conditions that utilize multiple cell types that mimic in vivo conditions is desired.

In one embodiment, the dispensing of the shaped organoid extrudates or the active cell complex is independently performed by manual or automated bioprinting. In one embodiment, the shaped organoid extrudates comprise a hydrogel, for example, the shaped organoid extrudates comprise Matrigel®, Cultrex® BME, CELLINK GelXA, CELLINK LAMININK 111, or any combination thereof. In one embodiment, the active cells comprise liver cells. In another embodiment, the active cell complex comprises a hydrogel, for example, the active cell complex comprises Matrigel®, Cultrex® BME, CELLINK GelXA, CELLINK LAMININK 111, or any combination thereof.

In one embodiment, multiple hydrogel structures can be deposited in a single well. These structures can vary in size, shape, material composition, and cell content. All subsequent analysis remains identical for multi-part constructs.

The shaped organoid extrudate herein may be deposited in a monolayer in any 2-dimensional shape configuration, including, but not limited to sheets, rings, or polygons. The shaped organoid extrudate may be polygonal, annular, ovular, elliptical, toroid, lemniscate, X-shaped, C-shaped, etc., and is not limited to any particular 2-dimensional shape (except as context may otherwise dictate). The shaped organoid extrudate may also by a manually seeded 3-dimensional shaped extrudate.

The shape of the shaped organoid extrudate is not limiting. FIGS. 1A-1B, FIGS. 2A-2B, FIGS. 3A-3B, FIG. 4, FIGS. 5A-5F, FIG. 6A-6B, FIGS. 7A-7C, FIG. 8, FIGS. 9A-9C, and FIGS. 10A-10B shows several examples of variations in shapes and sizes of MOI devices, well plates containing MOI devices, and wells not containing any MOI device.

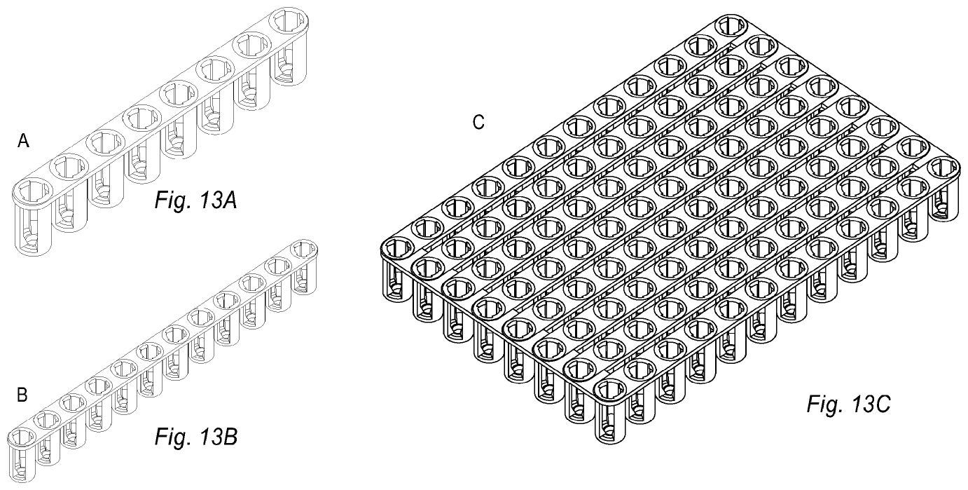

As shown in the figures herein, multiple MOIs may be arrayed together to fit into multiple wells, or all wells, of a plate, such that, for example, liver cells or tumor cells can be loaded into the carriers or platforms of multiple devices and then immersed into wells having different conditions, e.g., different therapeutic agents, serial dilutions of agents, etc. FIG. 13A-C depicts elevated perspective views of multiple MOI devices connected to span multiple wells of a well plate. FIG. 13A depicts a linear 8-well embodiment. FIG. 13B depicts a linear 12-well embodiment. FIG. 13C depicts a two-dimensional 96-well array to be used with a 96-well plate. Thus, an array comprising wo or more devices disclosed herein may be provided.

As used herein, the singular form “a”, “an” and “the” include plural references unless the context clearly dictates otherwise.

Throughout this application, various embodiments of the present disclosure may be presented in a range format. It should be understood that the description in range format is merely for convenience and brevity and should not be construed as an inflexible limitation on the scope disclosed herein. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Unless otherwise defined, all technical and/or scientific terms used herein have the same meaning as commonly understood by one of ordinary skill in the art to which the disclosure pertains. Although methods and materials similar or equivalent to those described herein can be used in the practice or testing of embodiments, exemplary methods and/or materials are described below. In case of conflict, the patent specification, including definitions, will control. In addition, the materials, methods, and examples are illustrative only and are not intended to be necessarily limiting. Each literature reference or other citation referred to herein is incorporated herein by reference in its entirety.

In the description presented herein, each of the steps and variations thereof are described. This description is not intended to be limiting and changes in the components, sequence of steps, and other variations would be understood to be within the scope of the present disclosure.

It is appreciated that certain features, which are, for clarity, described in the context of separate embodiments, may also be provided in combination in a single embodiment. Conversely, various features, which are, for brevity, described in the context of a single embodiment, may also be provided separately or in any suitable subcombination or as suitable in any other described embodiment. Certain features described in the context of various embodiments are not to be considered essential features of those embodiments unless the embodiment is inoperative without those elements.

Various embodiments and aspects of the present disclosure as delineated hereinabove and as claimed in the claims section below find experimental support in the following examples.

While certain features have been illustrated and described herein, many modifications, substitutions, changes, and equivalents will now occur to those of ordinary skill in the art. It is, therefore, to be understood that the appended claims are intended to cover all such modifications and changes as fall within the true spirit of the present disclosure.

### Example 1

### Mini-Organ Insert

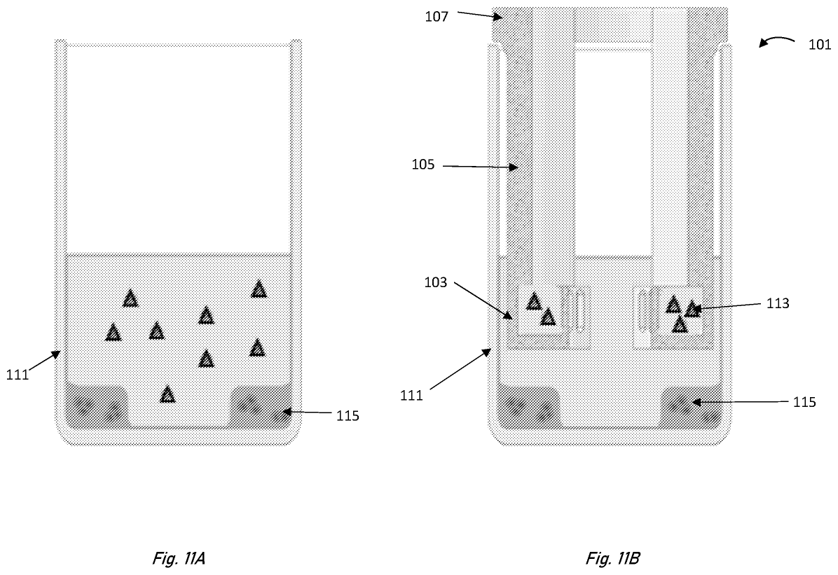

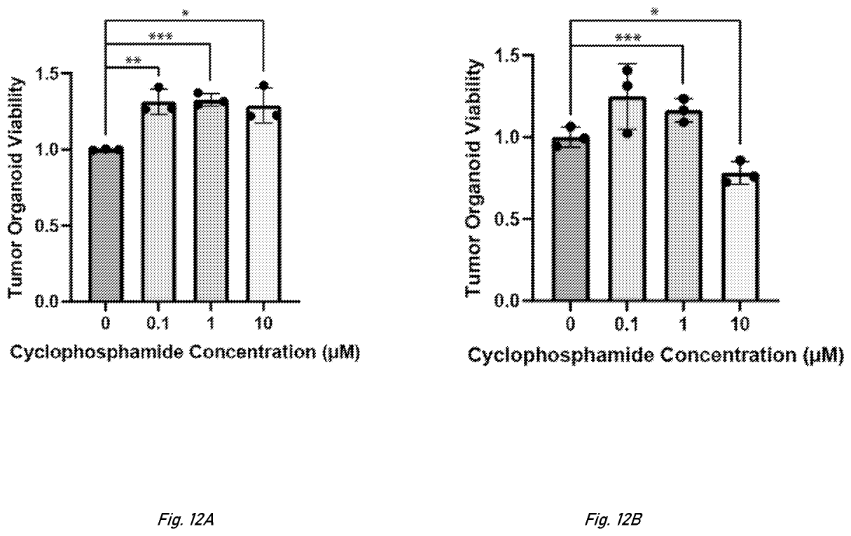

Insert devices as described herein were 3D printed in accordance with the descriptions in FIGS. 1A-1B, FIGS. 2A-2B, FIGS. 3A-3B, FIG. 4, FIGS. 5A-5F, FIG. 6A-6B, FIGS. 7A-7C, FIG. 8, FIGS. 9A-9C, and FIGS. 10A-10B. FIGS. 1A-1B present two preferred embodiments of the mini-organ insert (MOI) device. The MOI device is designed to fit into the well of either a standard 384-well plate, 96-well plate, 24-well plate, 12-well plate or 6-well plate, rest on the top rim and suspend a carrier or platform comprising cells in the culture medium; such cells may metabolize a therapeutic agent present in the medium. FIG. 1A shows one design and use of the MOI device. The 3D-printed insert comprises an annular carrier or platform suspended by three struts, into which carrier or platform a culture of cells (e.g., liver cells, intestinal cells or immune cells) are seeded in a hydrogel (e.g., in Matrigel®). Thus, the annular carrier or platform supports a ring of cells, e.g., liver organoid, which, using the MOI device, may be readily submerged in culture medium in a well-plate well. The MOI having the suspended carrier or platform of cells is then placed into a well having at its base a pre-printed shaped organoid extrudate or 2D monolayer or other cell aggregates; metabolic activity of either the carrier or platform, well cells, or both may modulate the therapeutic activity of therapeutic agents being tested on tumors, to simulate the bioactivity of the therapeutic agent in vivo. This method is compatible with existing drug screening protocols. In one embodiment, hepatocytes are bioprinted into the carrier or platform pockets of a 3D-printed well insert, which is then inserted into a tissue culture well. FIG. 11B shows a cross-sectional schematic view of a tissue culture well containing sarcoma tumor organoids and a MOI device containing hepatocytes.

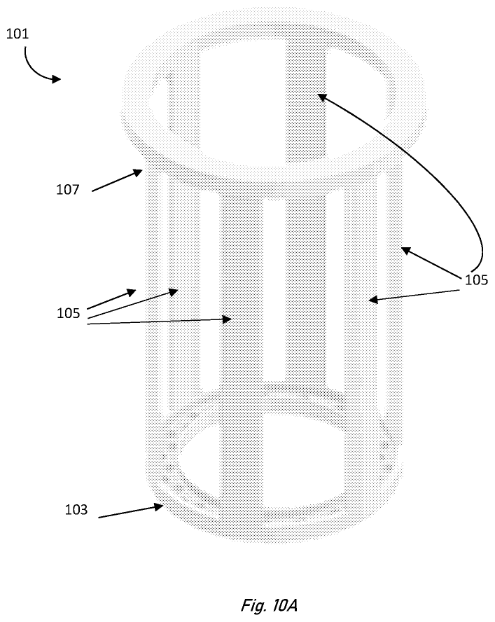

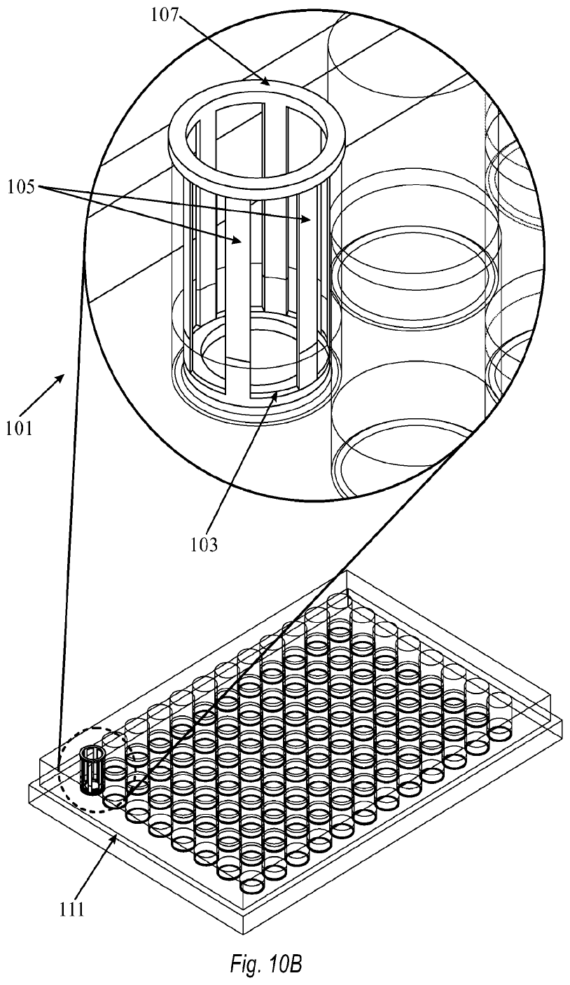

FIGS. 10A-10B depict another variation on the MOI, wherein the carrier or platform comprises a ring configuration to hold cells. The bottom of the ring is perforated to enhance interaction with the culture medium. The carrier or platform is suspended from the ring with vertical struts. As with the other embodiments of the MOI device, this variation of the MOI is designed to fit into the well, rest on the top rim and suspend a carrier or platform comprising cells within the medium. FIGS. 10A-10B show examples of the design and use. The 3D-printed insert comprises a ring carrier or platform at the bottom of each strut, into which cells (e.g., liver cells) are seeded (e.g., in Matrigel®). The MOI device is inserted into a well which may have at its base a pre-printed shaped organoid extrudate; metabolic activity of the carrier or platform modulates the therapeutic activity of agents being tested on tumors, to simulate the bioactivity of the therapeutic agent in vivo.

### Example 2

### Bioprinting Organoids for High-Throughput Drug Screening and Therapy Selection

Currently, a protocol for screening therapies on patient-derived sarcoma organoids relies upon manual deposition of the cell-laden gel around the perimeter of a well. This procedure is laborious and requires a refined technique thus significantly limits both the number of patients that can be tested as well as the number of drugs to be screened for each patient. Additionally, subsequent imaging and analysis of the organoids is complicated by inconsistencies in the focal planes of interest and meniscus effects generated by the well geometry.

Once the protocol has been validated, the tumor organoids can be implemented in drug screening process for patients. These patients can be selected based on the available amount of tissue, as concurrent screening using existing methods can also be conducted for comparison. The organoids can be incubated for 72 hours with bright field images taken every 24 hours. After the incubation period, the liquid in the wells is removed and replaced with fresh medium containing the drug of interest. All drugs can be tested with a minimum of 4 replicates and at concentrations of, for example, 0.1, 1, and 10 μM. Treatment is repeated twice over consecutive days. In one embodiment, a drug arrays containing nearly 500 compounds including standard chemotherapeutics and targeted therapies (e.g., kinase, PARP, proteasome, and HDAC inhibitors) can be used. After treatment, an ATP assay (CellTiter-Glo 3D, Promega) on each sample is performed to measure sensitivity.

In one embodiment, the bioprinting conditions can also be optimized using patient-derived cells. Materials and printing parameters may be varied. For example, the ATP assay is a surrogate for viability and biased toward drugs affecting metabolic pathways. Thus, a label-free image-based approach can be developed to quantify drug effects using morphological analysis. Similar methods have been developed for the characterization of single cells and organoids. Standard image analysis based on binary masking and morphological characterization can be performed using MATLAB, Python or other programming languages, and parameters such as area, circularity, and optical density can be correlated with viability to identify significant metrics. A neural network can be trained to predict the viability of the organoids by training the model on bright field images and extracted feature characteristics. This will leverage the existing database of sarcoma organoid images (collected by inventors) labeled with their sensitivity to therapy (viability after treatment). The overall goal of this analytical tool is to eliminate the need to perform a disruptive chemical assay to evaluate the effects of drugs on the organoids.

As described herein, the use of an insert device in which active cells from an organ such as liver cells or immune cells, provides a feature of in vitro screening that introduces a metabolic component into the evaluation of the potential effectiveness of a therapeutic agent. Rather than simply evaluate therapeutic agents or combinations against tumor cells to identify a potentially efficacious treatment, the added presence of, in one embodiment, liver cells that metabolize therapeutic agents, will provide better screening conditions. In some embodiments, the liver cells may inactivate a therapeutic agent, thus the bioassay using liver cells may indicate that a therapeutic agent, while effective against tumor cells, may not be bioavailable in vivo. In another embodiment, liver and other cells may activate therapeutic agents or prodrugs thereof to enhance their therapeutic value. Co-culture of the tumor cells and liver cells as described herein may identify effective therapeutic agents that would otherwise be considered inactive in vitro.

### General Procedures

In one embodiment, the screening process starts with harvesting patient-derived tissue samples that are dissociated into a single cell suspension upon mincing and treatment with collagenase IV. The cell suspension is then filtered through a 40 μm cell strainer. The cell suspension is mixed into a gel precursor solution and deposited in a ring or other shape around the perimeter of individual wells manually or automatedly, to provide a shaped organoid extrudate. Once the solution has gelled in a warm environment, medium is added. After 48-72 hours, the medium is replaced with medium containing the drugs of interest for screening. Images of each well are taken every 24 hours until the end of the experiment. Viability is tracked using a chemical assay and supplemented with image analysis. All media exchanges are managed by an automated fluid handler and imaging can also be managed by an automated imaging system. Variations of the foregoing that achieve the same or similar results are fully embraced herein.

In one embodiment, the term “hydrogels” encompasses both commercially available (e.g., CELLINK Series, Allevi Liver dECM) and lab-developed materials (e.g. GelMA, ColMA) derived from natural (e.g., collagen, gelatin, alginate) or synthetic biocompatible polymers (e.g. Pluronic, PEG, PPO). These materials are referred to herein as bioink.

In one embodiment, the Matrigel®-based bioink is a complex of Matrigel® and cell culture medium. Several ratios can be used when mixing a variety of gel matrices (e.g., Matrigel®, BME, etc.) with cell culture media (e.g., MammoCult, RPMI, DMEM). For example, 3 parts of medium is used to 4 parts of Matrigel®. In other embodiments, the ratio of medium to Matrigel® can be 1:2, 1:1, 2:1, 3:1 or pure Matrigel® solutions can be used. In other embodiments, a thickening agent such as xanthan gum or a cellulose derivative such as carboxymethylcellulose may be included at from about 1% to about 20% to modify the mechanical properties.

### Cell Seeding Requirements

In one embodiment, cells are seeded in a range of 2,000-10,000 cells per well in a 96-well plate or 10,000-200,000 cells per well in a 24-well plate. Cell density is dependent on a range of factors but is primarily determined by the amount of tissue harvested from the patient.

### Shape Requirements

The printed shape can be any polygon composed of a single or multiple layers. Multiple shapes can be printed within the same well. Each shape/structure can contain singular, multiple, or no cell types. Shapes must be able to be circumscribed by a circle with the internal diameter of the given well plate and must not occupy the central region of the well, for example, within a 0.5 mm radius of the center. In one embodiment, culture wells with a 6.35 mm diameter base can be used and any printing shapes can be used provided that the outer edge of the printing needle remains within the 6.35 mm circular boundary and outside of the excluded central radius. In one embodiment, a ring is printed having a mean diameter of 3.3 mm, and a width of 1 mm. As printed in the center of the well, the printed ring does not contact any wall of the well.

### Example 3

### A Portable Mini-Organ Insert (MOI) Using Liver Cells for Testing Liver-Metabolized Therapies on Tumor Organoids

The present example describes use of the mini-organ insert (MOI) device, screening drugs alone or in combination that would better inform clinicians on their suitability for the patient of interest. For purpose of illustration, the present example describes screening drugs for bone sarcoma. The methodology described herein would be equally applicable to screening other drugs for other cancer or disease.

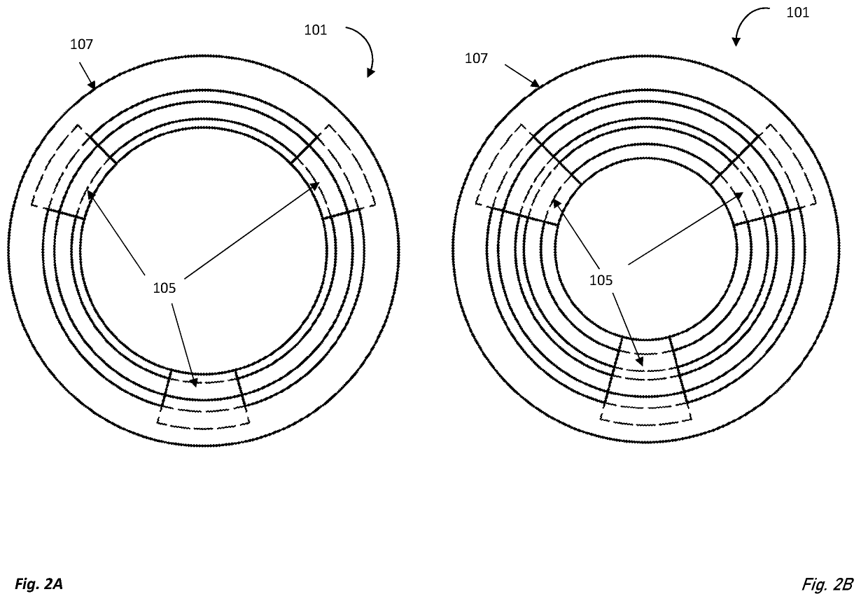

A bioprinting-based platform may be herein to create the organoids comprising sarcoma cells. In some examples, HepaRG™ cells (BIOPREDIC International), for example, can be printed in similar hydrogel constructs into a well insert, the mini-organoid insert (MOI, see FIG. 1A-1B, FIGS. 2A-2B, FIGS. 3A-3B, FIG. 4, FIGS. 5A-5F, FIG. 6A-6B, FIGS. 7A-7C, FIG. 8, FIGS. 9A-9C, and FIGS. 10A-10B). In one embodiment, the well insert is 3D printed using a stereolithography printer (Form 3, Formlabs). In one embodiment, the Surgical Guide Resin (Formlabs) can be used due to its biocompatibility and autoclave-safe properties. This material is also compatible with inverted microscopy due to its translucency. The MOI system allows direct immersion of the hepatocyte-seeded gels into the well containing the sarcoma organoids while also maintains an automation-compatible format by keeping cells away from the center of the well.

In other embodiments, the devices may be fabricated out of any biocompatible material such as a plastic or resin, for example, a medical grade of a photopolymer, PVC, polyethylene, PEEK, polycarbonate, polypropylene, etc., and by any method such as 3D printing, injection molding, blow molding, vacuum casting, thermoforming, assembly from extruded components, etc.

The MOI can be validated for all drugs of interest prior to applying it to sarcoma organoids. In one embodiment, the validation process includes printing 1,000-20,000 hepatocytes in the well insert and incubating in medium for 72 hours. In some embodiments, between about 5,000 and about 10,000 hepatocytes are provided in the well insert. At the 0.5, 1, 2, 4, 8, 12, 24, 48 and 72 h time-points, culture medium is collected from the well. Liquid chromatography mass spectrometry can be used to quantify the parent compound, its metabolites, and their concentrations. The goal is to identify conditions that recreate in vivo drug metabolism. For example, it is expected that 25-50% of ifosfamide will be converted into chloro-acetaldehyde, while the remainder would be converted to 4-hydroxy-ifosfamide and its more stable deactivated forms. To optimize these parameters, the size, shape, and cell density of the well insert can be changed if necessary. Hepatocyte viability and proliferation can be assessed for each time-point by Calcein AM and propidium iodide staining as well as an ATP assay. Optimal hepatocyte conditions and incubation periods for each drug can then be used in subsequent experiments.

To perform drug screening, the well insert can be studied in co-culture with cell lines that are sensitive to ifosfamide in mice, for example, EW-7 (Ewing's sarcoma) and SAOS-LM7 (osteosarcoma) cell lines. The first goal is to ensure that the well insert is not toxic to the sarcoma cells in the absence of a drug; this can be verified with viability assays. Next, whether and when phenotypic changes in the sarcoma cells occur due to interactions with the HepaRG cells are determined. IHC and H&E staining as well as RNAseq can be used to characterize changes between cells cultured with and without the well insert. In one embodiment, sarcoma organoids and the HepRG cells printed with the CELLINK BioX are incubated independently for the first 72 hours. After 3 days, the medium is replaced with drug-containing medium. Once the medium has been replaced, the well insert with the hepatocyte gel is transferred into the sarcoma organoid well. Every 24 hours thereafter the sarcoma organoids can be imaged, and medium replaced. After two treatments, the well insert can be removed, and organoid viability can be assayed as described herein.

Once the system has been validated on cell line-derived organoids, the screening can proceed to testing organoids established from bone sarcoma patient samples. Specimens from sarcoma patients have been collected and biobanked over the years. Existing databases can be mined to identify patients found to be clinically sensitive and patients resistant to drugs such as ifosfamide. Organoid sensitivity in the presence and absence of the well insert can be compared with the patient's clinical response. A correlation between in vitro response and patient radiologic and histopathological response to therapy is expected. The mini well insert platform described herein can be incorporated into existing drug screening protocol to screen commonly used front-line therapies. Employing the well insert platform described herein, a list of potential useful drugs can be provided to physicians who select treatment regimens as well as open the possibility to study the biological effects of these drugs in vitro.

What is claimed is:

1. A device for use with a tissue culture plate comprising a plurality of tissue culture wells, the device comprising a cylindrical well insert configured to be inserted into said tissue culture wells, wherein said well insert comprises a rim section on one end of the insert and one or more carrier or platform sections on an opposite end of the insert, wherein said carrier or platform section or sections is disposed around a perimeter of said opposite end, and said carrier or platform section or sections is positionable in said tissue culture wells so as to be suspended off the bottom of said tissue culture wells.

2. The device of claim 1, wherein said tissue culture plate is a 384-well plate or a 96-well plate or a 48-well plate or a 24-well plate or a 12-well plate or a 6-well plate.

3. The device of claim 1, wherein the distance between said rim section and said carrier or platform section is less than the depth of said tissue culture wells.

4. The device of claim 1, wherein said carrier or platform section is suspended off the bottom of said tissue culture wells by about 1 mm.

5. The device of claim 1, wherein said rim section has a diameter slightly larger than a diameter of said tissue culture wells.

6. The device of claim 1, wherein the carrier or platform section is configured to accommodate a cell complex.

7. The device of claim 6, wherein said cell complex is positioned not to occupy a central region of said tissue culture wells.

8. The device of claim 6, wherein said carrier or platform section comprises one or more annular carriers or platforms.

9. The device of claim 8, wherein said carrier or platform section comprises one annular carrier or platform.

10. The device of claim 8, wherein said carrier or platform section comprises two concentric annular carriers or platforms.

11. The device of claim 6, wherein said carrier or platform section comprises micropores and short vertical walls to keep said cell complex in place.

12. The device of claim 8, wherein each of said micropores have a size of about 100 microns to about 1,000 microns.

13. The device of claim 6, wherein said cell complex comprises a hydrogel.

14. The device of claim 6, wherein said cell complex comprises a hydrogel such as alginate, collagen, gelatin, or a basement membrane extract such as Matrigel®, Cultrex® BME, CELLINK GelXA, CELLINK LAMININK 111, or any combination thereof.

15. The device of claim 1, wherein said well insert comprises a wall that comprises open lattice or does not comprise any open space.

16. A system for use in screening candidate therapeutic agents for activities against tumor cells, the system comprising (i) a tissue culture plate comprising a plurality of tissue culture wells, and (ii) the device of claim 1.

17. The system of claim 16 wherein the tissue culture plate is a 384-well plate or a 96-well plate or a 48-well plate or a 24-well plate or a 12-well plate or a 6-well plate.

18. The system of claim 16, wherein each of said tissue culture wells comprises a tumor cell complex comprising tumor cells, said tumor cell complex is deposited as a shaped extrudate around a perimeter at a bottom of said tissue culture wells.

19. The system of claim 16, wherein said tumor cells are sarcoma cells or carcinoma cells or normal tissue cells.

20. The system of claim 16, wherein said tumor cell complex comprises a hydrogel.

21. The system of claim 16, wherein said tumor cell complex comprises a hydrogel such as alginate, collagen, gelatin, or a basement membrane extract such as Matrigel®, Cultrex® BME, CELLINK GelXA, CELLINK LAMININK 111, or any combination thereof.

22. The system of claim 16, wherein the carrier or platform section of the device of claim 1 comprises an active cell complex comprising cells that can metabolize one or more candidate therapeutic agents.

23. The system of claim 22, wherein said active cell complex comprises liver cells.

24. The system of claim 22, wherein said active cell complex comprises a hydrogel.

25. The system of claim 22, wherein said active cell complex comprises a hydrogel such as alginate, collagen, gelatin, or a basement membrane extract such as Matrigel®, Cultrex® BME, CELLINK GelXA, CELLINK LAMININK 111, or any combination thereof.

26. The system of claim 16, wherein each of said devices comprises a tumor cell complex comprising tumor cells.

27. The system of claim 26, wherein said tumor cells are sarcoma cells or carcinoma cells.

28. The system of claim 26, wherein said tumor cell complex comprises a hydrogel.

29. The system of claim 28, wherein said tumor cell complex comprises a hydrogel such as alginate, collagen, gelatin, or a basement membrane extract such as Matrigel®, Cultrex® BME, CELLINK GelXA, CELLINK LAMININK 111, or any combination thereof.

30. The system of claim 26, wherein the tissue culture well comprises an active cell complex comprising cells that can metabolize one or more candidate therapeutic agents.

31. The system of claim 30, wherein said active cell complex comprises liver cells.

32. The system of claim 30, wherein said active cell complex comprises a hydrogel.

33. The system of claim 32, wherein said active cell complex comprises a hydrogel such as alginate, collagen, gelatin, or a basement membrane extract such as Matrigel®, Cultrex® BME, CELLINK GelXA, CELLINK LAMININK 111, or any combination thereof.

34. A method for identifying therapeutic agents or combination thereof for treating a tumor in a patient, comprising the steps of
i) obtaining a sample of tumor cells optionally in single cell suspension, or as aggregates or clusters from the tumor of the patient;
ii) dispensing shaped organoid extrudates comprising said tumor cells into tissue culture wells;
iii) dispensing an active cell complex comprising active cells that can metabolize said therapeutic agents or combination thereof onto the carrier or platform section of the device of claim 1; and
iv) inserting the device of claim 1 into said tissue culture wells, and culturing said tumor cells and said active cells in said tissue culture wells with said therapeutic agents or combination thereof, wherein reduced growth, reduced mobility, altered biological properties or reduced viability of the tumor cells in the presence of said therapeutic agents or combination thereof identifies the therapeutic agents or combination thereof for treating the tumor in the patient.

35. A method for treating a tumor in a patient, comprising the steps of
i) obtaining a sample of tumor cells optionally in single cell suspension, or as aggregates or clusters from the tumor of the patient;
ii) dispensing shaped organoid extrudates comprising said tumor cells into tissue culture wells;
iii) dispensing an active cell complex comprising active cells that can metabolize said therapeutic agents or combination thereof onto the carrier or platform section of the device of claim 1;
iv) inserting the device of claim 1 into said tissue culture wells, and culturing said tumor cells and said active cells in said tissue culture wells with said therapeutic agents or combination thereof, wherein reduced growth, reduced mobility, altered biological properties, or viability of the tumor cells in the presence of said therapeutic agents or combination thereof identifies the therapeutic agents or combination thereof for treating the tumor in the patient; and
v) treating said patient with said therapeutic agents or combination thereof.

36. The method of claim 26 or 27, wherein the dispensing of the shaped organoid extrudates or the active cell complex is independently performed by manual or automated bioprinting.

37. The method of claim 26 or 27, wherein said tumor cells are sarcoma cells.

38. The method of claim 26 or 27, wherein said shaped organoid extrudates comprise a hydrogel.

39. The method of claim 26 or 27, wherein said shaped organoid extrudates comprise a basement membrane extract such as alginate, collagen, gelatin, Matrigel®, Cultrex® BME, CELLINK GelXA, CELLINK LAMININK 111, or any combination thereof.

40. The method of claim 26 or 27, wherein said active cells comprise liver cells.

41. The method of claim 26 or 27, wherein said active cell complex comprises a hydrogel.

42. The method of claim 26 or 27, wherein said active cell complex comprises a basement membrane extract such as alginate, collagen, gelatin, Matrigel®, Cultrex® BME, CELLINK GelXA, CELLINK LAMININK 111, or any combination thereof.

43. A method for identifying therapeutic agents or combination thereof for treating a tumor in a patient, comprising the steps of
i) obtaining a sample of tumor cells optionally in single cell suspension, or as aggregates or clusters from the tumor of the patient;
ii) providing said tumor cells into tissue culture wells;
iii) dispensing an active cell complex comprising active cells that can metabolize said therapeutic agents or combination thereof onto the carrier or platform section of the device of claim 1;
iv) introducing one or more therapeutic agents into the tissue culture wells, and culturing said tumor cells in said tissue culture wells with said therapeutic agents, wherein reduced growth, reduced mobility, altered biological properties, or reduced viability of the tumor cells in the presence of said therapeutic agents identifies the therapeutic agents or combination thereof for treating the tumor in the patient.

44. A method for treating a tumor in a patient, comprising the steps of
i) obtaining a sample of tumor cells optionally in single cell suspension, or as aggregates or clusters from the tumor of the patient;
ii) providing said tumor cells into tissue culture wells;
iii) dispensing an active cell complex comprising active cells that can metabolize said therapeutic agents or combination thereof onto the carrier or platform section of the device of claim 1;
iv) introducing one or more therapeutic agents into the tissue culture wells, and culturing said tumor cells in said tissue culture wells with said therapeutic agents, wherein reduced growth, reduced mobility, altered biological properties or reduced viability of the tumor cells in the presence of said therapeutic agents identifies the therapeutic agents or combination thereof for treating the tumor in the patient; and
(v) treating said patient with said therapeutic agents or combination thereof.

45. The method of claim 35 or 36, wherein said tumor cells are sarcoma or carcinoma cells.

46. The method of claim 35 or 36, wherein said active cells comprise liver cells or intestinal cells.

47. The method of claim 35 or 36, wherein said active cell complex comprises a hydrogel.

48. The method of claim 35 or 36, wherein said active cell complex comprises a hydrogel such as alginate, collagen, gelatin, or a basement membrane extract such as Matrigel®, Cultrex® BME, CELLINK GelXA, CELLINK LAMININK 111, or any combination thereof.

49. A method for identifying therapeutic agents or combination thereof for treating a tumor in a patient, comprising the steps of
i) obtaining a sample of tumor cells optionally in single cell suspension, or as aggregates or clusters from the tumor of the patient;
ii) dispensing said tumor cells onto the carrier or platform section of the device of claim 1;
iii) optionally dispensing an active cell complex comprising active cells that can metabolize said therapeutic agents or combination thereof into tissue culture wells; and
iv) inserting the device into said tissue culture wells, and culturing said tumor cells and optionally said active cells in said tissue culture wells with said therapeutic agents or combination thereof, wherein reduced growth, reduced mobility, altered biological properties or reduced viability of the tumor cells in the presence of said therapeutic agents or combination thereof identifies the therapeutic agents or combination thereof for treating the tumor in the patient.

50. A method for treating a tumor in a patient, comprising the steps of
i) obtaining a sample of tumor cells optionally in single cell suspension, or as aggregates or clusters from the tumor of the patient;
ii) dispensing said tumor cells onto the carrier or platform section of the device of claim 1;
iii) optionally dispensing an active cell complex comprising active cells that can metabolize said therapeutic agents or combination thereof into tissue culture wells;
iv) inserting the device of claim 1 into said tissue culture wells, and culturing said tumor cells and optionally said active cells in said tissue culture wells with said therapeutic agents or combination thereof, wherein reduced growth, reduced mobility, altered biological properties, or viability of the tumor cells in the presence of said therapeutic agents or combination thereof identifies the therapeutic agents or combination thereof for treating the tumor in the patient; and
v) treating said patient with said therapeutic agents or combination thereof.

51. The method of claim 49 or 50, wherein the dispensing of the tumor cells or the active cell complex is independently performed by manual or automated bioprinting.

52. The method of claim 49 or 50, wherein said tumor cells are sarcoma cells.

53. The method of claim 49 or 50, wherein said tumor cells are provided in a hydrogel.

54. The method of claim 53, wherein said hydrogel is alginate, collagen, gelatin, or a basement membrane extract such as Matrigel®, Cultrex® BME, CELLINK GelXA, CELLINK LAMININK 111, or any combination thereof.

55. The method of claim 4 or 50, wherein said active cells comprise liver cells.

56. The method of claim 49 or 50, wherein said active cell complex comprises a hydrogel.

57. The method of claim 56, wherein said hydrogel is alginate, collagen, gelatin, or a basement membrane extract such as Matrigel®, Cultrex® BME, CELLINK GelXA, CELLINK LAMININK 111, or any combination thereof.

58. A method for identifying therapeutic agents or combination thereof for treating a tumor in a patient, comprising the steps of
i) obtaining a sample of tumor cells optionally in single cell suspension, or as aggregates or clusters from the tumor of the patient;
ii) providing said tumor cells into tissue culture wells;
iii) dispensing an active cell complex comprising active cells that can metabolize said therapeutic agents or combination thereof onto the carrier or platform section of the device of claim 1;
iv) introducing one or more therapeutic agents into the tissue culture wells, and culturing said tumor cells in said tissue culture wells with said therapeutic agents, wherein reduced growth, reduced mobility, altered biological properties, or reduced viability of the tumor cells in the presence of said therapeutic agents identifies the therapeutic agents or combination thereof for treating the tumor in the patient.

59. A method for treating a tumor in a patient, comprising the steps of
i) obtaining a sample of tumor cells optionally in single cell suspension, or as aggregates or clusters from the tumor of the patient;
ii) providing said tumor cells into tissue culture wells;
iii) dispensing an active cell complex comprising active cells that can metabolize said therapeutic agents or combination thereof onto the carrier or platform section of the device of claim 1;
iv) introducing one or more therapeutic agents into the tissue culture wells, and culturing said tumor cells in said tissue culture wells with said therapeutic agents, wherein reduced growth, reduced mobility, altered biological properties or reduced viability of the tumor cells in the presence of said therapeutic agents identifies the therapeutic agents or combination thereof for treating the tumor in the patient; and
v) treating said patient with said therapeutic agents or combination thereof.

60. The method of claim 58 or 59, wherein said tumor cells are sarcoma or carcinoma cells.

61. The method of claim 58 or 59, wherein said active cells comprise liver cells or intestinal cells.

62. The method of claim 58 or 59, wherein said active cell complex comprises a hydrogel.

63. The method of claim 58 or 59, wherein said active cell complex comprises a hydrogel such as alginate, collagen, gelatin, or a basement membrane extract such as Matrigel®, Cultrex® BME, CELLINK GelXA, CELLINK LAMININK 111, or any combination thereof.

64. A method for identifying a bioactive compound, comprising the steps of
i) obtaining a sample of normal or tumor cells optionally in single cell suspension, or as aggregates or clusters;
ii) dispensing said cells as 2D monolayer or 3D aggregates or 3D shaped organoid extrudates comprising said cells into tissue culture wells;
iii) dispensing an active cell complex comprising active cells that can metabolize said small molecules or biologics or combination thereof onto the carrier or platform section of the device of claim 1;
iv) inserting the device of claim 1 into said tissue culture wells, and culturing said cells and said active cells in said tissue culture wells with said small molecules or biologics or combination thereof, wherein reduced growth, reduced mobility, change in biological properties or viability of the tumor cells in the presence of said small molecules or biologics or combination thereof identifies the active agents or combination thereof for biological investigation.

65. A system for use in in vitro evaluation of a biological activities, the system comprising (i) a tissue culture plate comprising a plurality of tissue culture wells, and (ii) the device of claim 1.
